(19) United States
(12) Patent Application Publication
(10) Pub. No.: US 2023/0293593 A1
(43) Pub. Date: Sep. 21, 2023

(54) HYPOIMMUNOGENIC NEURAL CELLS FOR THE TREATMENT OF NEUROLOGICAL DISORDERS AND CONDITIONS

(71) Applicant: Sana Biotechnology, Inc., Seattle, WA (US)

(72) Inventors: Sonja SCHREPFER, South San Francisco, CA (US); Rebeca RAMOS-ZAYAS, Waltham, MA (US)

(21) Appl. No.: 17/907,084

(22) PCT Filed: Mar. 25, 2021
(86) PCT No.: PCT/US2021/024228

§ 371 Date: Sep. 22, 2022

Related U.S. Application Data
(60) Provisional application No. 62/994,750, filed on Mar. 25, 2020.

(51) Int. Cl. A61K 35/30; A61P 25/28; C12N 5/0793; C12N 5/079; A61P 25/16

(52) U.S. Cl. CPC A61K 35/30; A61P 25/16; A61P 25/28; C12N 5/0619; C12N 5/0622; C12N 2506/45

(57) ABSTRACT

Disclosed herein are cells including neural cells that evade immune recognition such as microglial response and related methods of their use and generation. In some embodiments, the cells disclosed herein have reduced levels or activities of MHC I and/ or MHC II human leukocyte antigens, and in some instances, exogenously express CD47. In some embodiments, the cells are derived from pluripotent stem cells that evade immune recognition by a recipient subject.

This application claims priority under 35 U.S.C. § 119(e) to U.S. Provisional Application No. 62/994,750 filed Mar. 25, 2020, the disclosure of which is herein incorporated by reference in its entirety.

### BACKGROUND

Neurological disorders encompass numerous afflictions such as acute brain injury such as stroke, head injury, and cerebral palsy; spinal cord injury; neurodegenerative diseases such as Alzheimer’s disease and Parkinson’s disease; and a large number of central nervous system dysfunctions such as depression, epilepsy, and schizophrenia. In fact, stroke is the leading cause of adult disability and the third cause of death worldwide.

There is substantial evidence in both animal models and human patients that neural cell transplantation is a scientifically feasible and clinically promising approach to the treatment of neurological disorders and conditions.

There remains a need for novel approaches, compositions and methods for producing cell-based therapies that avoid detection by the recipient’s immune system.

### BRIEF SUMMARY

Provided herein is a method for inhibiting microglial phagocytosis of a population of neural cells administered in a patient comprising administering to the patient a therapeutically effective amount of a population of neural cells comprising an exogenous CD47 polypeptide and reduced expression of MHC class I and/or MHC class II human leukocyte antigens.

In some embodiments, the population of neural cells comprises reduced expression of MHC class I or MHC class II human leukocyte antigens. In some embodiments, the population of neural cells comprises reduced expression of MHC class I and MHC class II human leukocyte antigens.

In some embodiments, the administering comprises grafting the population of neural cells into the patient’s central or peripheral nervous system. In many embodiments, the grafting comprises injecting the population of neural cells into the patient. In numerous embodiments, the grafting comprises disrupting the patient’s blood-brain barrier.

In some embodiments, the population of neural cells exhibits long-term survival after the disruption of the patient’s blood-brain barrier. In various embodiments, the population of neural cells exhibits long-term function after the disruption of the patient’s blood-brain barrier.

In some embodiments, the population of neural cells maintains long-term survival in the patient after the patient experiences a subsequent disruption of the patient’s blood-brain barrier that is secondary to a neurological disorder or condition. In many embodiments, the population of neural cells maintains long-term function in the patient after the patient experiences a subsequent disruption of the patient’s blood-brain barrier that is secondary to a neurological disorder or condition. In some embodiments, the subsequent disruption of the patient’s blood-brain barrier is due to an infection or a stroke.

In a number of embodiments, the population of neural cells survives and/or functions in the patient for at least one month, two months, three months, four months or more after administration.

In some embodiments, the patient is not administered an immunosuppressive agent before administration of the population of neural cells. In many embodiments, the patient is not administered an immunosuppressive agent after administration of the population of neural cells. In certain embodiments, the patient requires a reduced level of immunosuppression or is substantially free of immunosuppression.

In some embodiments, the neural cell is selected from the group consisting of a cerebral endothelial cell, a neuron, an ependymal cell, an astrocyte, a microglial cell, an oligodendrocyte, a Schwann cell, a progenitor thereof, and a precursor thereof.

In several embodiments, the neural cell is a neural progenitor cell. In various embodiments, the neural cell is a glial progenitor cell. In many embodiments, the neural cell is a neuronal progenitor cell.

In certain embodiments, the microglial phagocytosis is associated with a neurological disorder or condition.

In some embodiments, the neurological disorder or condition is selected from the group consisting of stroke, amyotrophic lateral sclerosis (ALS), cerebral hemorrhage, Parkinson’s disease, epilepsy, spinal cord injury, childhood hereditary leukodystrophies, congenital dysmyelination, Pelizaeus-Merzbacher disease, metabolic leukodystrophies, vanishing white matter disease, adrenoleukodystrophy, Canavan’s Disease, lysosomal storage diseases, Tay-Sachs disease, Sandhoff’s disease, Krabbe’s disease, Batten’s disease, metachromatic leukodystrophy, cerebral palsy, periventricular leukomalacia, spastic diplegias of prematurity, age-related white matter loss, subcortical dementia, vascular Leukoencephalopathies, subcortical stroke, diabetic leukoencephalopathy, hypertensive leukoencephalopathy, spinal cord injury, autoimmune demyelination, progressive multiple sclerosis, transverse myelitis, inflammatory demyelination, radiation toxicity, neurodegenerative diseases, Huntington’s Disease, frontotemporal dementia, and cerebrovascular disorders. In some embodiments, the neurological disorder or condition is Pelizaeus-Merzbacher disease. In certain embodiments, the neurological disorder or condition is progressive multiple sclerosis. In many embodiments, the neurological disorder or condition is Huntington’s Disease.

In some embodiments, the population of neural cells express CD47 at a higher level than in an unmodified pluripotent cell or in a unmodified neural cell.

In some embodiments, the population of neural cells express a suicide gene that is activated by a trigger that causes the neural cell to die.

Provided herein is a method for inhibiting microglial phagocytosis of a population of neural cells administered in a patient comprising administering to the patient a therapeutically effective amount of a population of neural cells comprising an exogenous CD47 polypeptide and reduced expression of B2M and/or CIITA.

In some embodiments, the population of neural cells comprises reduced expression of B2M or CIITA. In some embodiments, the population of neural cells comprises reduced expression of B2M and CIITA.

In some embodiments, the administering comprises grafting the population of neural cells into the patient’s central or peripheral nervous system. In numerous embodiments, the grafting comprises injecting the population of neural cells into the patient. In many embodiments, the grafting comprises disrupting the patient’s blood-brain barrier.

In many embodiments, the population of neural cells exhibits long-term survival after the disruption of the patient’s blood-brain barrier. In numerous embodiments, the population of neural cells exhibits long-term function after the disruption of the patient’s blood-brain barrier.

In some embodiments, the population of neural cells maintains long-term survival in the patient after the patient experiences a subsequent disruption of the patient’s blood-brain barrier that is secondary to a neurological disorder or condition.

In some embodiments, the population of neural cells maintains long-term function in the patient after the patient experiences a subsequent disruption of the patient’s blood-brain barrier that is secondary to a neurological disorder or condition. In other embodiments, the subsequent disruption of the patient’s blood-brain barrier is due to an infection or a stroke.

In many embodiments, the population of neural cells survives and/or functions in the patient for at least one month, two months, three months, four months or more after administration.

In some embodiments, the patient is not administered an immunosuppressive agent before administration of the population of neural cells. In certain embodiments, the patient is not administered an immunosuppressive agent after administration of the population of neural cells. In some embodiments, the patient requires a reduced level of immunosuppression or is substantially free of immunosuppression.

In some embodiments, the neural cell is selected from the group consisting of a cerebral endothelial cell, a neuron, an ependymal cell, an astrocyte, a microglial cell, an oligodendrocyte, a Schwann cell, a progenitor thereof, and a precursor thereof. In some embodiments, the neural cell is a neural progenitor cell. In various embodiments, the neural cell is a glial progenitor cell. In certain embodiments, the neural cell is a neuronal progenitor cell.

In some embodiments, the microglial phagocytosis is associated with a neurological disorder or condition.

In some embodiments, the neurological disorder or condition is selected from the group consisting of stroke, amyotrophic lateral sclerosis (ALS), cerebral hemorrhage, Parkinson’s disease, epilepsy, spinal cord injury, childhood hereditary leukodystrophies, congenital dysmyelination, Pelizaeus-Merzbacher disease, metabolic leukodystrophies, vanishing white matter disease, adrenoleukodystrophy, Canavan’s Disease, lysosomal storage diseases, Tay-Sachs disease, Sandhoff’s disease, Krabbe’s disease, Batten’s disease, metachromatic leukodystrophy, cerebral palsy, periventricular leukomalacia, spastic diplegias of prematurity, age-related white matter loss, subcortical dementia, vascular Leukoencephalopathies, subcortical stroke, diabetic leukoencephalopathy, hypertensive leukoencephalopathy, spinal cord injury, autoimmune demyelination, progressive multiple sclerosis, transverse myelitis, inflammatory demyelination, radiation toxicity, neurodegenerative diseases, Huntington’s Disease, frontotemporal dementia, and cerebrovascular disorders. In some embodiments, the neurological disorder or condition is Pelizaeus-Merzbacher disease. In many embodiments, the neurological disorder or condition is progressive multiple sclerosis. In various embodiments, the neurological disorder or condition is Huntington’s Disease.

In several embodiments, the population of neural cells express CD47 at a higher level than in a parental pluripotent stem cell (e.g., an unmodified pluripotent stem cell) or in a unmodified neural cell. In some embodiments, the population of neural cells express a suicide gene that is activated by a trigger that causes the neural cell to die.

In many embodiments, the population of neural cells are glial progenitor cells.

Provided herein is an in-vitro method for producing a therapeutically effective amount of a population of human neural cells from a population of human pluripotent stem cells comprising the steps of a) genetically modifying human pluripotent stem cells to i) reduce expression of MHC class I human leukocyte antigens and/or MHC class II human leukocyte antigens in the human pluripotent stem cells and ii) overexpress an exogenous CD47 polypeptide in the human pluripotent stem cells, b) differentiating the human pluripotent stem cells into neural cells; and c) assaying the neural cells for a hypoimmunogenicity phenotype and/or one or more neural cell-specific markers, gene expression, or gene expression profile.

In some embodiments, the step a) further comprises genetically modifying human pluripotent stem cells to reduce expression of MHC class I and MHC class II human leukocyte antigens.

In some embodiments, the step a) further comprises genetically modifying human pluripotent stem cells to reduce expression of MHC class I and MHC class II human leukocyte antigens.

In some embodiments, the human pluripotent stem cells of step a)ii) express CD47 at a level higher than in the population of human pluripotent stem cells before step a).

In some embodiments, the human neural cells of step b) or c) express CD47 at a level higher than in an unmodified neural cell or a neuronal cell not genetically modified by step a) .

In some embodiments, the human neural cells of step b) or c) have reduced expression of MHC class I human leukocyte antigens and/or MHC class II human leukocyte antigens compared to an unmodified human neural cell or a neuronal cell not genetically modified by step a).

In some embodiments, the step a) further comprises iii) express a suicide gene in the human pluripotent stem cells.

In some embodiments, the assaying of the human neural cells in step c) comprises assaying for the hypoimmunogenicity phenotype by Elispot, ELISA, FACS, PCR, or mass cytometry (CYTOF).

Provided is an isolated neural cell comprising an exogenous CD47 polypeptide and reduced expression of MHC class I and/or class II human leukocyte antigens, wherein the cell evades immune recognition when administered to a patient.

In some embodiments, the isolated neuronal cell further comprises reduced expression of MHC class I and class II human leukocyte antigens.

In some embodiments, the isolated neural cell is selected from the group consisting of a cerebral endothelial cell, a neuron, an ependymal cell, an astrocyte, a microglial cell, an oligodendrocyte, a Schwann cell, a progenitor thereof, and a precursor thereof. In some embodiments, the isolated neural cell is a neural progenitor cell. In many embodiments, the isolated neural cell is a glial progenitor cell. In numerous embodiments, the isolated neural cell is a neuronal progenitor cell. In some embodiments, the isolated neural cell is a cerebral endothelial cell. In various embodiments, the isolated neural cell is a dopamine neuron.

In some embodiments, the isolated neural cell evades immune recognition in vitro. In many embodiments, the isolated neural cell evades immune recognition when grafted into a patient’s central or peripheral nervous system.

In some embodiments, the isolated neural cell undergoes, exhibits, or stimulates reduced microglial phagocytosis in vitro. In many embodiments, the isolated neural cell undergoes, exhibits, or stimulates reduced microglial phagocytosis when grafted into a patient’s central nervous system.

In some embodiments, the isolated neural cell has reduced expression of B2M and/or CIITA.

In some embodiments, isolated neural cell comprises one or more CD47 transgenes.

In some embodiments, the expression of the one or more CD47 transgene is controlled by constitutive promoters. In certain embodiments, expression of the one or more CD47 transgene is controlled by neuronal specific promoters.

In some embodiments, provided is a composition comprising a population of any of the isolated neural cells of the present technology.

In some embodiments, the composition further comprises a pharmaceutically acceptable carrier.

Also, provided herein is method for treating a neurological disorder or condition in a patient comprising administering to the patient a therapeutically effective amount of a population of neural cells comprising an exogenous CD47 polypeptide and reduced expression of MHC class I human leukocyte antigens and/or MHC class I human leukocyte antigens, wherein population of the neural cells undergoes, exhibits, or stimulates reduced microglial phagocytosis upon administration.

In some embodiments, the administering comprises grafting the population of neural cells into the patient’s central or peripheral nervous system. In some embodiments, the grafting comprises injecting the population of neural cells into the patient. In various embodiments, the grafting comprises disrupting the patient’s blood-brain barrier.

In some embodiments, the population of neural cells exhibits long-term survival after the disruption of the patient’s blood-brain barrier. In many embodiments, the population of neural cells exhibits long-term function after the disruption of the patient’s blood-brain barrier.

In some embodiments, the population of neural cells maintains long-term survival in the patient after the patient experiences a subsequent disruption of the patient’s blood-brain barrier that is secondary to the neurological disorder or condition. In many embodiments, the population of neural cells maintains long-term function in the patient after the patient experiences a subsequent disruption of the patient’s blood-brain barrier that is secondary to the neurological disorder or condition.

In some embodiments, the subsequent disruption of the patient’s blood-brain barrier is due to an infection or a stroke.

In some embodiments, the population of neural cells survives and/or functions in the patient for at least one month, two months, three months, four months or more after administration.

In some embodiments, the patient is not administered an immunosuppressive agent before, during, and/or after administration of the population of neural cells. In some embodiments, the patient requires a reduced level of immunosuppression or is substantially free of immunosuppression.

In some embodiments, the neural cell is selected from the group consisting of a cerebral endothelial cell, a neuron, an ependymal cell, an astrocyte, a microglial cell, an oligodendrocyte, a Schwann cell, a progenitor thereof, and a precursor thereof. In various embodiments, the neural cell is a neural progenitor cell. In some embodiments, the neural cell is a glial progenitor cell. In certain embodiments, the neural cell is a neuronal progenitor cell.

In many embodiments, the neurological disorder or condition is selected from the group consisting of stroke, amyotrophic lateral sclerosis (ALS), cerebral hemorrhage, Parkinson’s disease, epilepsy, spinal cord injury, childhood hereditary leukodystrophies, congenital dysmyelination, Pelizaeus-Merzbacher disease, metabolic leukodystrophies, vanishing white matter disease, adrenoleukodystrophy, Canavan’s Disease, lysosomal storage diseases, Tay-Sachs disease, Sandhoff’s disease, Krabbe’s disease, Batten’s disease, metachromatic leukodystrophy, cerebral palsy, periventricular leukomalacia, spastic diplegias of prematurity, age-related white matter loss, subcortical dementia, vascular Leukoencephalopathies, subcortical stroke, diabetic leukoencephalopathy, hypertensive leukoencephalopathy, spinal cord injury, autoimmune demyelination, progressive multiple sclerosis, transverse myelitis, inflammatory demyelination, radiation toxicity, neurodegenerative diseases, Huntington’s Disease, frontotemporal dementia, and cerebrovascular disorders. In some embodiments, the neurological disorder or condition is Pelizaeus-Merzbacher disease. In many embodiments, the neurological disorder or condition is progressive multiple sclerosis. In some embodiments, the neurological disorder or condition is Huntington’s Disease.

Provided herein is a neural cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the neural cell undergoes, exhibits, or stimulates reduced microglial phagocytosis.

In some embodiments, the neural cell is selected from the group consisting of a cerebral endothelial cell, a neuron, an ependymal cell, an astrocyte, a microglial cell, an oligodendrocyte, a Schwann cell, a progenitor thereof, and a precursor thereof. In some embodiments, the neural cell is a neural progenitor cell. In many embodiments, the neural cell is a glial progenitor cell. In various embodiments, the neural cell is a neuronal progenitor cell.

Provided herein is a cerebral endothelial cell in vitro differentiated from a stem cell expressing an exogenous CD47 polypeptide and expressing i) reduced expression levels of MHC class I and/or II human leukocyte antigens and/or ii) reduced expression levels of B2M and/or CIITA, wherein the cerebral endothelial cell undergoes, exhibits, or stimulates reduced microglial phagocytosis. In some embodiments, the cell forms vasculature when administered to a patient’s brain.

Provided herein is a microglial cell in vitro differentiated from a stem cell expressing an exogenous CD47 polypeptide and expressing: i)reduced expression levels of MHC class I and/or II human leukocyte antigens and/or ii) reduced expression levels of B2M and/or CIITA, wherein the microglial cell undergoes, exhibits, or stimulates reduced microglial phagocytosis.

Provided herein is an oligodendrocyte in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the oligodendrocyte undergoes, exhibits, or stimulates reduced microglial phagocytosis.

Provided herein is a Schwann cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the Schwann cell undergoes, exhibits, or stimulates reduced microglial phagocytosis.

Provided herein is an astrocyte in vitro differentiated from a stem cell expressing an exogenous CD47 polypeptide and expressing: i)reduced expression levels of MHC class I and/or II human leukocyte antigens and/or ii) reduced expression levels of B2M and/or CIITA, wherein the astrocyte undergoes, exhibits, or stimulates reduced microglial phagocytosis.

Provided herein is an ependymal cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the ependymal cell undergoes, exhibits, or stimulates reduced microglial phagocytosis.

Provided herein is a neuron in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis. In some embodiments, the neuron is a dopamine neuron.

Provided herein is a dopamine neuron in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

Provided herein is an isolated neural cell comprising an exogenous CD24 polypeptide and reduced expression of MHC class I human leukocyte antigens and/or MHC class II human leukocyte antigens, wherein the isolated neural cell evades immune recognition when administered to a patient.

In some embodiments, the isolated neural cell is selected from the group consisting of a cerebral endothelial cell, a neuron, an ependymal cell, an astrocyte, a microglial cell, an oligodendrocyte, a Schwann cell, a progenitor thereof, and a precursor thereof. In some embodiments, the isolated neural cell is a neural progenitor cell. In various embodiments, the isolated neural cell is a glial progenitor cell. In many embodiments, the isolated neural cell is a neuronal progenitor cell. In many embodiments, the neural cell is a cerebral endothelial cell.

In some embodiments, the neural cell evades immune recognition in vitro. In certain embodiments, the neural cell evades immune recognition when grafted into a patient’s central or peripheral nervous system. In some embodiments, the neural cell undergoes, exhibits, or stimulates reduced microglial phagocytosis in vitro. In many embodiments, the neural cell undergoes, exhibits, or stimulates reduced microglial phagocytosis when grafted into a patient’s central nervous system.

In some embodiments, the neural cell has reduced expression of B2M and/or CIITA. In many embodiments, the neural cell comprises one or more CD24 transgenes. In some embodiments, expression of the one or more CD47 transgene is controlled by constitutive promoters. In certain embodiments, expression of the one or more CD47 transgene is controlled by neuronal specific promoters.

Also provided is a composition comprising a population of any of the isolated neural cells of described herein. In some embodiments, further comprises a pharmaceutically acceptable carrier.

Provided is a method for treating a neurological disorder or condition in a patient comprising administering to the patient a therapeutically effective amount of a population of neural cells comprising an exogenous CD24 polypeptide and reduced expression of MHC class I and/or MHC class II human leukocyte antigens.

In some embodiments, the method further comprises reduced expression of MHC class I and MHC class II human leukocyte antigens.

In some embodiments, the administering comprises grafting the population of neural cells into the patient’s central or peripheral nervous system. In certain embodiments, the grafting comprises injecting the population of neural cells into the patient. In some embodiments, the grafting comprises disrupting the patient’s blood-brain barrier.

In some embodiments, the population of neural cells maintains long-term survival after the disruption of the patient’s blood-brain barrier. In certain embodiments, the population of neural cells maintains long-term function after the disruption of the patient’s blood-brain barrier.

In some embodiments, the population of neural cells maintains long-term survival in the patient after the patient experiences a subsequent disruption of the patient’s blood-brain barrier that is secondary to the neurological disorder or condition.

In some embodiments, the population of neural cells maintains long-term function in the patient after the patient experiences a subsequent disruption of the patient’s blood-brain barrier that is secondary to the neurological disorder or condition.

In some embodiments, the subsequent disruption of the patient’s blood-brain barrier is due to an infection or a stroke.

In some embodiments, the population of neural cells survives and/or functions in the patient for at least one month, two months, three months, four months or more after administration.

In various embodiments, the patient is not administered an immunosuppressive agent before administration of the population of neural cells. In certain embodiments, the patient is not administered an immunosuppressive agent after administration of the population of neural cells. In many embodiments, the patient requires a reduced level of immunosuppression or is substantially free of immunosuppression. In some embodiments, the neural cell is selected from the group consisting of a cerebral endothelial cell, a neuron, an ependymal cell, an astrocyte, a microglial cell, an oligodendrocyte, a Schwann cell, a progenitor thereof, and a precursor thereof. In various embodiments, the neural cell is a neural progenitor cell. In many embodiments, the neural cell is a glial progenitor cell. In numerous embodiments, the neural cell is a neuronal progenitor cell.

In some embodiments, the neurological disorder or condition is selected from the group consisting of stroke, amyotrophic lateral sclerosis (ALS), cerebral hemorrhage, Parkinson’s disease, epilepsy, spinal cord injury, childhood hereditary leukodystrophies, congenital dysmyelination, Pelizaeus-Merzbacher disease, metabolic leukodystrophies, vanishing white matter disease, adrenoleukodystrophy, Canavan’s Disease, lysosomal storage diseases, Tay-Sachs disease, Sandhoff’s disease, Krabbe’s disease, Batten’s disease, metachromatic leukodystrophy, cerebral palsy, periventricular leukomalacia, spastic diplegias of prematurity, age-related white matter loss, subcortical dementia, vascular Leukoencephalopathies, subcortical stroke, diabetic leukoencephalopathy, hypertensive leukoencephalopathy, spinal cord injury, autoimmune demyelination, progressive multiple sclerosis, transverse myelitis, inflammatory demyelination, radiation toxicity, neurodegenerative diseases, Huntington’s Disease, frontotemporal dementia, and cerebrovascular disorders. In various embodiments, the neurological disorder or condition is Pelizaeus-Merzbacher disease. In some embodiments, the neurological disorder or condition is progressive multiple sclerosis. In many embodiments, the neurological disorder or condition is Huntington’s Disease.

Provided is a neural cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neural cell undergoes, exhibits, or stimulates reduced microglial phagocytosis.

In some embodiments, the neural cell is selected from the group consisting of a cerebral endothelial cell, a neuron, an ependymal cell, an astrocyte, a microglial cell, an oligodendrocyte, a Schwann cell, a progenitor thereof, and a precursor thereof. In various embodiments, the neural cell is a neural progenitor cell. In many embodiments, the neural cell is a glial progenitor cell. In numerous embodiments, the neural cell is a neuronal progenitor cell.

Provided is a cerebral endothelial cell in vitro differentiated from a stem cell expressing an exogenous CD24 polypeptide and expressing: i) reduced expression levels of MHC class I and II human leukocyte antigens and/or ii) reduced expression levels of B2M and/or CIITA, wherein the cerebral endothelial cell undergoes, exhibits, or stimulates reduced microglial phagocytosis.

In some embodiments, the cell forms vasculature when administered to a patient’s brain.

Provided is a microglial cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the microglial cell undergoes, exhibits, or stimulates reduced microglial phagocytosis.

Provided is an oligodendrocyte in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the oligodendrocyte undergoes, exhibits, or stimulates reduced microglial phagocytosis.

Provided is a Schwann cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the Schwann cell undergoes, exhibits, or stimulates reduced microglial phagocytosis.

Provided is an astrocyte in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the astrocyte undergoes, exhibits, or stimulates reduced microglial phagocytosis.

Provided is an ependymal cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the ependymal cell undergoes, exhibits, or stimulates reduced microglial phagocytosis.

Provided is a neuron in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

In some embodiments, the neuron is a dopamine neuron.

Provided is a dopamine neuron in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

Provided herein is use of a neural cell to treat a neurological disease, comprising an isolated neural cell comprising an exogenous CD47 polypeptide and reduced expression of MHC class I and/or class II human leukocyte antigens, wherein the cell evades immune recognition when administered to a patient.

Provided herein is use of a neural cell to treat a neurological disease, comprising a neural cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the neural cell undergoes, exhibits, or stimulates reduced microglial phagocytosis.

Provided herein is use of a neural cell to treat a neurological disease, comprising a cerebral endothelial cell in vitro differentiated from a stem cell expressing an exogenous CD47 polypeptide and expressing i) reduced expression levels of MHC class I and/or II human leukocyte antigens and/or ii) reduced expression levels of B2M and/or CIITA, wherein the cerebral endothelial cell undergoes, exhibits, or stimulates reduced microglial phagocytosis.

Provided herein is use of a neural cell to treat a neurological disease, comprising a microglial cell in vitro differentiated from a stem cell expressing an exogenous CD47 polypeptide and expressing: i)reduced expression levels of MHC class I and/or II human leukocyte antigens and/or ii) reduced expression levels of B2M and/or CIITA, wherein the microglial cell undergoes, exhibits, or stimulates reduced microglial phagocytosis.

Provided herein is use of a neural cell to treat a neurological disease, comprising an oligodendrocyte in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the oligodendrocyte undergoes, exhibits, or stimulates reduced microglial phagocytosis.

Provided herein is use of a neural cell to treat a neurological disease, comprising a Schwann cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the Schwann cell undergoes, exhibits, or stimulates reduced microglial phagocytosis.

Provided herein is use of a neural cell to treat a neurological disease, comprising an astrocyte in vitro differentiated from a stem cell expressing an exogenous CD47 polypeptide and expressing: i)reduced expression levels of MHC class I and/or II human leukocyte antigens and/or ii) reduced expression levels of B2M and/or CIITA, wherein the astrocyte undergoes, exhibits, or stimulates reduced microglial phagocytosis.

Provided herein is use of a neural cell to treat a neurological disease, comprising an ependymal cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the ependymal cell undergoes, exhibits, or stimulates reduced microglial phagocytosis.

Provided herein is use of a neural cell to treat a neurological disease, comprising a neuron in vitro differentiated from a stem cell expressing an exogenous CD47 polypeptide and expressing: i)reduced expression levels of MHC class I and/or II human leukocyte antigens and/or ii) reduced expression levels of B2M and/or CIITA, wherein the neuron undergoes, exhibits, or stimulates reduced microglial phagocytosis.

Provided herein is use of a neural cell to treat a neurological disease, comprising a dopamine neuron in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

Provided herein is use of a neural cell to treat a neurological disease, comprising an isolated neural cell comprising an exogenous CD24 polypeptide and reduced expression of MHC class I human leukocyte antigens and/or MHC class II human leukocyte antigens, wherein the isolated neural cell evades immune recognition when administered to a patient.

Provided herein is use of a neural cell to treat a neurological disease, comprising a neural cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neural cell undergoes, exhibits, or stimulates reduced microglial phagocytosis.

Provided herein is use of a neural cell to treat a neurological disease, comprising a cerebral endothelial cell in vitro differentiated from a stem cell expressing an exogenous CD24 polypeptide and expressing: i) reduced expression levels of MHC class I and II human leukocyte antigens and/or ii) reduced expression levels of B2M and/or CIITA, wherein the cerebral endothelial cell undergoes, exhibits, or stimulates reduced microglial phagocytosis.

Provided herein is use of a neural cell to treat a neurological disease, comprising a microglial cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the microglial cell undergoes, exhibits, or stimulates reduced microglial phagocytosis.

Provided herein is use of a neural cell to treat a neurological disease, comprising an oligodendrocyte in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the oligodendrocyte undergoes, exhibits, or stimulates reduced microglial phagocytosis.

Provided herein is use of a neural cell to treat a neurological disease, comprising a Schwann cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the Schwann cell undergoes, exhibits, or stimulates reduced microglial phagocytosis.

Provided herein is use of a neural cell to treat a neurological disease, comprising an astrocyte in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the astrocyte undergoes, exhibits, or stimulates reduced microglial phagocytosis.

Provided herein is use of a neural cell to treat a neurological disease, comprising an ependymal cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the ependymal cell undergoes, exhibits, or stimulates reduced microglial phagocytosis.

Provided herein is use of a neural cell to treat a neurological disease, comprising a neuron in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

Provided herein is use of a neural cell to treat a neurological disease, comprising a dopamine neuron in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

In some embodiments, the neurological disorder or condition is selected from the group consisting of stroke, amyotrophic lateral sclerosis (ALS), cerebral hemorrhage, Parkinson’s disease, epilepsy, spinal cord injury, childhood hereditary leukodystrophies, congenital dysmyelination, Pelizaeus-Merzbacher disease, metabolic leukodystrophies, vanishing white matter disease, adrenoleukodystrophy, Canavan’s Disease, lysosomal storage diseases, Tay-Sachs disease, Sandhoff’s disease, Krabbe’s disease, Batten’s disease, metachromatic leukodystrophy, cerebral palsy, periventricular leukomalacia, spastic diplegias of prematurity, age-related white matter loss, subcortical dementia, vascular Leukoencephalopathies, subcortical stroke, diabetic leukoencephalopathy, hypertensive leukoencephalopathy, spinal cord injury, autoimmune demyelination, progressive multiple sclerosis, transverse myelitis, inflammatory demyelination, radiation toxicity, neurodegenerative diseases, Huntington’s Disease, frontotemporal dementia, and cerebrovascular disorders.

In some embodiments, the neurological disorder or condition is Pelizaeus-Merzbacher disease. In many embodiments, the neurological disorder or condition is progressive multiple sclerosis. In some embodiments, the neurological disorder or condition is Huntington’s Disease.

Provided herein is a dopamine neuron in vitro differentiated from a stem cell, wherein the dopamine neuron expresses an exogenous CD47 polypeptide, wherein the dopamine neuron has reduced expression levels of B2M and CIITA, and wherein the dopamine neuron undergoes, exhibits, or stimulates reduced microglial phagocytosis.

Provided herein is a dopamine neuron in vitro differentiated from a stem cell, wherein the dopamine neuron expresses an exogenous CD24 polypeptide, wherein the dopamine neuron has reduced expression levels of B2M and CIITA, and wherein the dopamine neuron undergoes, exhibits, or stimulates reduced microglial phagocytosis.

Provided herein is a glial progenitor cell in vitro differentiated from a stem cell, wherein the glial progenitor cell expresses an exogenous CD47 polypeptide, wherein the glial progenitor cell has reduced expression levels of B2M and CIITA, and wherein the glial progenitor cell undergoes, exhibits, or stimulates reduced microglial phagocytosis or evades microglial phagocytosis.

Provided herein is a glial progenitor cell in vitro differentiated from a stem cell, wherein the glial progenitor cell expresses an exogenous CD24 polypeptide, wherein the glial progenitor cell has reduced expression levels of B2M and CIITA, and wherein the glial progenitor cell undergoes, exhibits, or stimulates reduced microglial phagocytosis or evades microglial phagocytosis.

Detailed descriptions of hypoimmunogenic cells, methods of producing thereof, and methods of using thereof are found in WO2016183041 filed May 9, 2015 and WO2018132783 filed Jan. 14, 2018, the disclosures including the sequence listings, figures and examples are incorporated herein by reference in their entirety.

### BRIEF DESCRIPTION OF THE DRAWINGS

FIG. 1 shows wild-type (wt) human ECs, double knockout (B2M-/-CIITA-/-) ECs, and double knockout/CD47Tg (B2M-/-CIITA-/- CD47 tg; dKO/CD47Tg) ECs. The ECs were co-cultured with allogeneic human macrophages or microglia to look at the effect of CD47 expression on microglia inhibition. CD47 protected dKO cells from macrophage killing. CD47 also seemed to protect dKO/CD47Tg cells from microglia phagocytosis.

FIG. 2 shows a comparison of immune response after allogeneic miPSC-derived endothelial transplantation (e.g., wild-type ECs or dKO/CD47Tg ECs) into the brain versus muscle, as determined by IFN-gamma Elispot. Injections of wt cells into the brain induced a T cell response. Injection of the dKO/CD47Tg cells seemed to not induce a systemic T cell response (which was seen in other organs).

FIG. 3 shows a comparison of immune response after allogeneic miPSC-derived endothelial transplantation (e.g., wild-type ECs or dKO/CD47Tg ECs) into the brain versus muscle, as determined by donor specific antibodies (DSA). Injections of wt cells into the brain induced a donor-specific antibody response. Injections of dKO/CD47Tg cells did not induce DSA production (which was seen in other organs).

FIG. 4 depicts bioluminescence imaging of wild-type ECs were transplanted into the striatum of healthy allogeneic BALB/c mice brains. Mouse iPSC-derived endothelial cells were transplanted into the striatum of healthy allogeneic BALB/c mice. 4 out of 5 animals rejected the wt ECs within 19 days.

FIG. 5 depicts bioluminescence imaging of wild-type mouse iPSC-derived endothelial cells (ECs) were transplanted into the striatum of healthy immunocompromised SCID-beige mice brains. The wt cells were detected in immunocompetent scid beige mice from day 0 (d0) to day 11 (d11).

FIG. 6 depicts bioluminescence imaging of dKO/CD47Tg (B2M-/-CIITA-/- CD47 tg) ECs that were transplanted into the striatum of healthy allogeneic BALB/c mice brains. Mouse iPSC-derived endothelial cells were transplanted into the striatum of healthy allogeneic BALB/c mice.

FIG. 7 depicts bioluminescence imaging of dKO/CD47Tg (B2M-/-CIITA-/- CD47 tg) ECs that were transplanted into the striatum of healthy immunocompromised SCID-beige mice brains. miPSC-derived endothelial cells were transplanted into the striatum of healthy scid beige mice.

FIG. 8A and FIG. 8B depict differences in microglia and macrophage killing of dKO cells. FIG. 8A shows the effect on human or mouse dKO (B2M-/-CIITA-/-) cells co-cultured with either allogeneic human macrophages, human microglia, or mouse microglia. The experiments assessed the effect of the absence of CD47 on macrophage and microglia mediated killing. Allogeneic macrophages and microglia sensed and killed the dKO cells. FIG. 8B shows the effect on human or mouse dKO (B2M-/-CIITA-/-) cells co-cultured with either xenogeneic (cross-species) human macrophages, human microglia or mouse microglia assessed the effect on macrophage and microglia mediated killing. Xenogeneic microglia did not sense nor killed the dKO cells. Specifically, human microglia did not kill the mouse dKO cells and mouse microglia did not kill the human dKO cells.

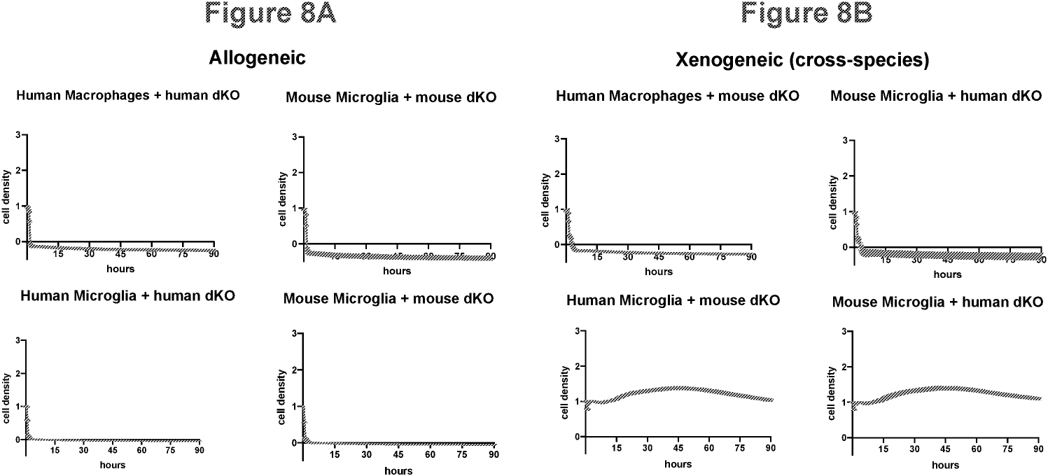

FIG. 9 are a set of representative FACS plots showing Nanog, Oct4 and Sox 2 expression in wild-type iPSCs, dKO/CD47Tg iPSCs (“1-B4 bulk CD47”) and dKO iPSCs (“1-B4 dKO) as described in Example 6. Expression of Nanog, Oct4 and Sox 2 in non-pluripotent control cells (HEK293 cells) is also shown in the plots.

FIG. 10 are a set of representative FACS plots showing FoxA2, Otx2, Nkx6.1, Nkx2.1, Nkx2.2, Soxl, and Pax6 expression in dopamine progenitor cells differentiated from either wild-type iPSCs, dKO/CD47Tg iPSCs (“1-B4 bulk CD47”), or dKO iPSCs (“1-B4 dKO”) as described in Example 6. Expression of the markers in the corresponding control iPSCs is also shown in the plots.

FIG. 11 are a set of representative FACS plots showing CD47 expression in wild-type iPSCs, dKO/CD47Tg iPSCs, dopamine progenitor cells differentiated from such wild-type iPSCs, and dopamine progenitor cells differentiated from such dKO/CD47Tg iPSCs, as described in Example 6. Fold change in CD47 levels is provided in the figure.

FIG. 12A and FIG. 12B depict bar graphs of forkhead box protein A2 (FoxA2), LIM homeobox transcription factor 1 (LMX1A), and (nuclear receptor related 1) Nurrl gene expression in dopamine neurons and maturing neurons differentiated from either wild-type and dKO/CD47Tg iPSCs, as described in Example 6.

FIG. 13 depicts immunofluorescence imaging of maturing neurons differentiated from either wild-type and dKO/CD47Tg iPSCs, as described in Example 6. The cells were stained to detect FoxA2, tyrosine hydroxylase (TH), pituitary homeobox (Pitx3), engrailed-1 (EN1), and BarH-like 1 homeobox protein (Barhl). Nuclei were detected using DAPI staining.

Other objects, advantages and embodiments of the present technology will be apparent from the detailed description following.

### DETAILED DESCRIPTION

### I. Introduction

The present technology is related to neural cells derived from stem cells and their use to treat neurological disorders and conditions. Also, the present technology utilizes methods for producing neural cell types from other non-neural cell types, including, but not limited to, pluripotent stem cells, induced pluripotent stem cells (iPSCs), and the like.

To overcome the problem of a recipient subject’s immune rejection of cell-derived and/or tissue transplants, the inventors have developed and disclose herein a neural cell (including precursor cells and progenitor cells thereof) that is immune-evasive. Such neural cells are not rejected by the recipient subject’s immune system. In many instances, the neural cells evade macrophage phagocytosis and/or microglia phagocytosis upon transplantation into recipient subject. In numerous instances, the hypoimmunogenic neural cells engraft into the recipient subject’s nervous system, such as the central and peripheral nervous systems, and are not eliminated by way of either macrophage or microglia phagocytosis, or both.

In some embodiments, neural hypoimmunogenic cells of the present technology are not subject to an innate immune cell rejection. In various embodiments, the neural hypoimmunogenic cells are not susceptible to NK cell-mediated lysis. In many embodiments, the neural hypoimmunogenic cells are not susceptible to macrophage engulfment. In many embodiments, the neural hypoimmunogenic cells are not susceptible to microglial engulfment. In some embodiments, the neural hypoimmunogenic cells maintain engrafted in the recipient subject after disturbance or disruption of the recipient’s blood-brain barrier. In some embodiments, neural hypoimmunogenic cells are useful as a source of universally compatible cells or tissues (e.g., universal donor cells or tissues) that can be transplanted into a recipient subject with little to no immunosuppressant agent needed. Such neural hypoimmunogenic cells retain cell-specific characteristics and features upon transplantation.

In some embodiments, provided herein are stem cells or neural cells differentiated therefrom that evade immune recognition and rejection after administration to an MHC-mismatched allogenic recipient. In some instances, the neural cells produced from the stem cells of the present technology evade immune rejection when administered (e.g., transplanted or grafted) to MHC-mismatched allogenic recipient. In other words, the stem cells and neural cells derived from such stem cells are hypoimmunogenic. In some embodiments, hypoimmunogenic stem cells and neural cells thereof of the present technology have reduced immunogenicity (such as, at least 2.5%-99% less immunogenicity) compared to corresponding wild-type cells. In some instances, the hypoimmunogenic stem cells lack immunogenicity compared to corresponding wild-type cells. Thus, the neural cells differentiated from such stem cells are suitable as universal donor cells for transplantation or engrafting into a recipient patient. In some embodiments, such cells are non-immunogenic to a human patient.

### II. Definitions

As described in the present technology, the following terms will be employed, and are defined as indicated below.

As used herein to characterize a cell, the term “hypoimmunogenic” generally means that such cell is less prone to immune rejection by a subject into which such cells are transplanted. For example, relative to an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. In some embodiments, genome editing technologies are used to modulate the expression of MHC I and MHC II genes, and thus, generate a hypoimmunogenic cell. In some embodiments, a hypoimmunogenic cell evades immune rejection in an MHC-mismatched allogenic recipient. In some instance, differentiated cells produced from the hypoimmunogenic stem cells evade immune rejection when administered (e.g., transplanted or grafted) to an MHC-mismatched allogenic recipient. In some embodiments, a hypoimmunogenic cell is protected from T cell-mediated adaptive immune rejection and/or innate immune cell rejection.

Hypoimmunogenicity of a cell can be determined by evaluating the immunogenicity of the cell such as the cell’s ability to elicit adaptive and innate immune responses. Such immune response can be measured using assays recognized by those skilled in the art. In some embodiments, an immune response assay measures the effect of a hypoimmunogenic cell on T cell proliferation, T cell activation, T cell killing, NK cell proliferation, NK cell activation, and macrophage activity. In some cases, hypoimmunogenic cells and derivatives thereof undergo decreased killing by T cells and/or NK cells upon administration to a subject. In some instances, the cells and derivatives thereof show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is non-immunogenic or fails to elicit an immune response in a recipient subject.

“Immunosuppressive factor” or “immune regulatory factor” or “tolerogenic factor” as used herein include hypoimmunity factors, complement inhibitors, and other factors that modulate or affect the ability of a cell to be recognized by the immune system of a host or recipient subject upon administration, transplantation, or engraftment.

“Safe harbor locus” as used herein refers to a gene locus that allows safe expression of a transgene or an exogenous gene. Exemplary “safe harbor” loci include a CCR5 gene, a CXCR4 gene, a PPP1R12C (also known as AAVS1) gene, an albumin gene, a SHS231 locus, a CLYBL gene, and a Rosa gene (e.g., ROSA26). A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation.

“Modulation” of gene expression refers to a change in the expression level of a gene. Modulation of expression can include, but is not limited to, gene activation and gene repression. Modulation may also be complete, i.e. wherein gene expression is totally inactivated or is activated to wildtype levels or beyond; or it may be partial, wherein gene expression is partially reduced, or partially activated to some fraction of wildtype levels.

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

“Pluripotent stem cells” as used herein have the potential to differentiate into any of the three germ layers: endoderm (e.g., the stomach linking, gastrointestinal tract, lungs, etc.), mesoderm (e.g., muscle, bone, blood, urogenital tissue, etc.) or ectoderm (e.g. epidermal tissues and nervous system tissues). The term “pluripotent stem cells,” as used herein, also encompasses “induced pluripotent stem cells”, or “iPSCs”, or a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al, Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

As used herein, the terms “grafting”, “administering,” “introducing”, “implanting” and “transplanting” as well as grammatical variations thereof are used interchangeably in the context of the placement of cells (e.g. cells described herein) into a subject, by a method or route which results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e. g. twenty-four hours, to a few days, to as long as several years. In some embodiments, the cells can also be administered (e.g., injected) a location other than the desired site, such as in the brain or subcutaneously, for example, in a capsule to maintain the implanted cells at the implant location and avoid migration of the implanted cells.

As used herein, the term “treating” and “treatment” includes administering to a subject an effective amount of cells described herein so that the subject has a reduction in at least one symptom of a disease (disorder or condition) or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a disease or disorder are alleviated by at least 5%, at least 10%, at least 20%, at least 30%, at least 40%, or at least 50% upon treatment of the disease. For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease. The term, as applied to an isolated cell, includes subjecting the cell to any kind of process or condition or performing any kind of manipulation or procedure on the cell. As applied to a subject, the term refers to administering a cell or population of cells in which a target polynucleotide sequence (e.g., B2M) has been altered ex vivo according to the methods described herein to an individual. The individual is usually ill or injured, or at increased risk of becoming ill relative to an average member of the population and in need of such attention, care, or management.

The term “neurological disorder” or “neurological condition” as used herein is defined as a disorder or condition that affects the central nervous system, the peripheral nervous system, or both, including any cells or tissues thereof. With respect to the inventive methods, cells and compositions, the neurological disorder or condition can be any neurological disorder or condition, including any of stroke, amyotrophic lateral sclerosis (ALS), cerebral hemorrhage, Parkinson’s disease, epilepsy, spinal cord injury, any cerebrovascular disorder, any adult neurological disorder or condition, any childhood neurological disorder or condition, any congenital neurological disorder or condition a hereditary leukodystrophy, congenital dysmyelination, Pelizaeus-Merzbacher Disease, any metabolic leukodystrophy, vanishing white matter disease, adrenoleukodystrophy, Canavan’s Disease, any lysosomal storage disease, Tay-Sachs disease, Sandhoff disease, Krabbe disease, Batten’s disease, metachromatic leukodystrophy, cerebral palsy, periventricular leukomalacia, spastic diplegias of prematurity, age-related white matter loss, subcortical dementia, any vascular leukoencephalopathy, subcortical stroke, diabetic leukoencephalopathy, hypertensive leukoencephalopathy, spinal cord injury, autoimmune demyelination, progressive multiple sclerosis, transverse myelitis, inflammatory demyelination, radiation toxicity, any neurodegenerative disease, Huntington’s Disease, ALS, frontotemporal dementia, and the like.

In additional or alternative embodiments, the present technology contemplates altering target polynucleotide sequences in any manner which is available to the skilled artisan, e.g., utilizing a nuclease system such as a TAL effector nuclease (TALEN) or zinc finger nuclease (ZFN) system. It should be understood that although examples of methods utilizing CRISPR/Cas (e.g., Cas9 and Cas12a) and TALEN are described in detail herein, the present technology is not limited to the use of these methods/systems. Other methods of targeting, e.g., B2M, to reduce or ablate expression in target cells known to the skilled artisan can be utilized herein.

The methods of the present technology can be used to alter a target polynucleotide sequence in a cell. The present technology contemplates altering target polynucleotide sequences in a cell for any purpose. In some embodiments, the target polynucleotide sequence in a cell is altered to produce an engineered cell. As used herein, an “engineered cell” or “modified cell” refers to a cell with a resulting genotype that differs from its original genotype. In some instances, an “engineered cell” exhibits an altered phenotype, for example when a normally functioning gene is altered using the CRISPR/Cas systems of the present technology. In other instances, an “engineered cell” exhibits a wild-type phenotype, for example when a CRISPR/Cas system of the present technology is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system can be used to induce an indel of any length or a point mutation in a target polynucleotide sequence.

As used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use the CRISPR/Cas systems to knock out a target polynucleotide sequence or a portion thereof based upon the details described herein.

In some embodiments, the alteration results in a knock out of the target polynucleotide sequence or a portion thereof. Knocking out a target polynucleotide sequence or a portion thereof using a CRISPR/Cas system can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

By “knock in” herein is meant a process that adds a genetic function to a host cell. This causes increased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

In some embodiments, an alteration or modification described herein results in reduced expression of a target or selected polynucleotide sequence. In some embodiments, an alteration or modification described herein results in reduced expression of a target or selected polypeptide sequence.

In some embodiments, an alteration or modification described herein results in increased expression of a target or selected polynucleotide sequence. In some embodiments, an alteration or modification described herein results in increased expression of a target or selected polypeptide sequence.

The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level or reference cell, for example a decrease by at least about 20%, or at least about 30%, or at least about 40%, or at least about 50%, or at least about 60%, or at least about 70%, or at least about 80%, or at least about 90% or up to and including a 100% decrease (i. e. absent level as compared to a reference sample), or any decrease between 10-100% as compared to a reference level or reference cell.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level or reference cell, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level or reference cell.

As used herein, the term “exogenous” in intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. An exogenous molecule includes a molecule, construct, factor and the like that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of neurons is an exogenous molecule with respect to an adult neuron cell. An exogenous molecule can comprise, for example, a functioning version of a malfunctioning endogenous molecule or a malfunctioning version of a normally-functioning endogenous molecule. An exogenous molecule or factor can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

The term “endogenous” refers to a referenced molecule or polypeptide that is present in the cell. Similarly, the term when used in reference to expression of an encoding nucleic acid refers to expression of an encoding nucleic acid contained within the cell and not exogenously introduced.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat′l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al, infra).

One example of an algorithm that is suitable for determining percent sequence identity and sequence similarity is the BLAST algorithm, which is described in Altschul et al, J. Mol. Biol. 215:403-410 (1990). Software for performing BLAST analyses is publicly available through the National Center for Biotechnology Information.

The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

It is noted that the claims may be drafted to exclude any optional element. As such, this statement is intended to serve as antecedent basis for use of such exclusive terminology as “solely,” “only,” and the like in connection with the recitation of claim elements or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the present technology. Any recited method may be carried out in the order of events recited or in any other order that is logically possible. Although any methods and materials similar or equivalent to those described herein may also be used in the practice or testing of the technology, representative illustrative methods and materials are now described.

Before the present technology is further described, it is to be understood that this technology is not limited to some embodiments described, as such may, of course, vary. It is also to be understood that the terminology used herein is for the purpose of describing some embodiments only, and is not intended to be limiting, since the scope of the present technology will be limited only by the appended claims.

Unless defined otherwise, all technical and scientific terms used herein have the same meaning as commonly understood by one of ordinary skill in the art to which the present technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the present technology. The upper and lower limits of these smaller ranges may independently be included in the smaller ranges and are also encompassed within the technology, subject to any specifically excluded limit in the stated range. Where the stated range includes one or both of the limits, ranges excluding either or both of those included limits are also included in the technology. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the present technology is not entitled to antedate such publication by virtue of prior technology. Further, the dates of publication provided might be different from the actual publication dates, which may need to be independently confirmed.

### III. Detailed Description of the Embodiments

### A. Hypoimmunogenic Neural Cells Derived From Pluripotent Stem Cells

Provided herein are hypoimmunogenic neural cells and neural progenitor cells including, but not limited to, cerebral endothelial cells, cerebral endothelial progenitor cells, neurons, neuronal progenitor cells, dopaminergic neurons, dopaminergic neuronal progenitor cells, glial cells, glial progenitor cells, ependymal cells, ependymal progenitor cells, astrocytes, astrocyte progenitor cells, microglial cells, microglial progenitor cells, oligodendrocytes, oligodendrocyte progenitor cells, Schwann cells, and Schwann progenitor cells. One skilled in the art would recognize that neural cells encompass each and every type of cell of the nervous system including those of each neural lineage. See, e.g., Ma et al., Cur Opin Neurobio, 2018, 50:7-16 for detailed descriptions of exemplary lineages of neural cells in the mammalian brain.

In some embodiments, any of the hypoimmunogenic neural cells described herein evade microglial phagocytosis. Microglia are a type of neuroglia (glial cell) located throughout the brain and spinal cord. As the resident macrophage-like cells, they act as the first and main form of active immune defense in the central nervous system (CNS). SIRPα is known to inhibit microglial phagocytic activity because the engulfment of myelin is augmented when microglial SIRPα is blocked with antibodies or knocked down by SIRPα-shRNA (Gitik et al., J Neuroinflammation. 2011 Mar 15; 8:24). This inhibitory property of SIRPα is thought to be critical for the maintenance of myelin integrity under normal conditions or following mild brain damage. However, the inhibitory property of SIRPα may be disadvantageous in massive brain injuries or degeneration when rapid clearance of damaged myelin is essential. Interactions between CD47 on myelin and SIRPα play a role in this pathway (see, e.g., Gitik et al., 2011). The cells described herein may be useful for engrafting into a recipient subject without inducing a neuroinflammatory response, such as a microglial response to the transplanted cells.

In some embodiments, the neural cells evade immune recognition and responses when administered to a subject. The cells can evade killing by immune cells in vitro and in vivo. In some embodiments, the cells evade killing by macrophages, microglial cells, and NK cells. In some embodiments, the cells are ignored by immune cells or a subject’s immune system, including the neuroinflammatory system. In some embodiments, the neural cells fail to recruit and/or activate microglial cells to the transplantation or engraftment site. In some embodiments, the neural cells fail to promote or induce proliferation of microglial cells at the transplantation or engraftment site. In some embodiments, microglial cells in the recipient subject do not infiltrate the transplantation or graft site containing the neural cells.

Methods of determining whether a neural cell evades immune recognition include, but are not limited to, IFN-y Elispot assays, microglia killing assays, cell engraftment animal models, cytokine release assays, ELISAs, killing assays using bioluminescence imaging or chromium release assay or Xcelligence® analysis, mixed-lymphocyte reactions, immunofluorescence analysis, etc.

The hypoimmunogenic neural cells are derived from pluripotent stem cells including, but not limited to, pluripotent stem cells and induced pluripotent stem cells that evade immune recognition or immune response by a recipient subject. Such hypoimmunogenic neural cells can avoid undergoing phagocytosis by neuroimmune cells in the body. In some embodiments, the hypoimmunogenic neural cells evade microglia-mediated killing of themselves upon administration into a recipient subject. In some embodiments, the hypoimmunogenic neural cells evade macrophage-mediated killing of themselves upon administration into a recipient subject. In some embodiments, the pluripotent stem cell is modified to exhibit reduced expression of MHC class I human leukocyte antigens. In other embodiments, the pluripotent stem cell is modified to exhibit reduced expression of MHC class II human leukocyte antigens. In some embodiments, the pluripotent stem cell is modified to exhibit reduced expression of MHC class I and II human leukocyte antigens. In some embodiments, the pluripotent stem cell is modified to exhibit reduced expression of MHC class I and/or II human leukocyte antigens and exhibit increased CD47 expression. In some instances, a cell overexpresses CD47 by harboring one or more CD47 transgenes. In some embodiments, the pluripotent stem cell is modified to exhibit reduced expression of MHC class I and/or II human leukocyte antigens and exhibit increased CD24 expression. In some instances, a cell overexpresses CD24 by harboring one or more CD24 transgenes. Such pluripotent stem cells are hypoimmunogenic pluripotent cells.

Any of the pluripotent stem cells of the present technology can be differentiated into neural cells. In some embodiments, the neural cells exhibit reduced expression of MHC class I and/or II human leukocyte antigens. In some instances, expression of MHC class I and/or II human leukocyte antigens is reduced compared to unmodified or wildtype neural cells. In some embodiments, the neural cells exhibit increased CD47 or CD24 expression. In some instances, expression of CD47 is increased compared to unmodified or wildtype neural cells. Methods for reducing levels of MHC class I and/or II human leukocyte antigens and increasing the expression of CD47 and CD24 are described herein.

The neural cells and progenitors thereof can be used to treat various neurological disorders and conditions.

In some embodiments, the hypoimmunogenic neural cells including, but not limited to, cerebral endothelial cells, neurons, dopamine neurons, glial cells, cerebral endothelial progenitor cells, neuronal progenitors, dopamine neuronal progenitors, and glial progenitor cells described herein are administered to a subject to ameliorate or treat stroke. In some embodiments, the neural cells and/or progenitors thereof are administered to a subject who has experienced a stroke.

In some embodiments, the hypoimmunogenic neural cells and/or progenitors thereof including, but not limited to, cerebral endothelial cells, neurons, dopamine neurons, glial cells, cerebral endothelial progenitor cells, neuronal progenitors, dopamine neuronal progenitors, and glial progenitor cells described herein are administered to a subject to alleviate a symptom or effect of amyotrophic lateral sclerosis (ALS). In some embodiments, the neurons, glial cells and/or progenitors thereof are administered to a subject with ALS.

In some embodiments, the hypoimmunogenic neural cells and/or progenitors thereof including, but not limited to, cerebral endothelial cells, neurons, dopamine neurons, glial cells, cerebral endothelial progenitor cells, neuronal progenitors, dopamine neuronal progenitors, and glial progenitor cells described herein are administered to a subject to alleviate a symptom or effect of cerebral hemorrhage. In some embodiments, cerebral endothelial cells and/or progenitors thereof are administered to a subject who has experienced a cerebral hemorrhage.

In some embodiments, the hypoimmunogenic neural cells and/or progenitors thereof including, but not limited to, cerebral endothelial cells, neurons, dopamine neurons, glial cells, cerebral endothelial progenitor cells, neuronal progenitors, dopamine neuronal progenitors, and glial progenitor cells described herein are administered to a subject to alleviate a symptom or effect of Parkinson’s disease. In some embodiments, dopaminergic neurons and/or progenitors thereof are administered to a patient with Parkinson’s disease.

In some embodiments, the hypoimmunogenic neural cells and/or progenitors thereof including, but not limited to, cerebral endothelial cells, neurons, dopamine neurons, noradrenergic neurons, GABAergic interneurons, glial cells, cerebral endothelial progenitor cells, neuronal progenitors, dopamine neuronal progenitors, noradrenergic neuronal progenitors, GABAergic interneuron progenitors. and glial progenitor cells described herein are administered to a subject to alleviate a symptom or effect of an epileptic seizure. In some embodiments, noradrenergic neurons, GABAergic interneurons and/or progenitors thereof are administered to a patient who has experienced an epileptic seizure.

In some embodiments, the hypoimmunogenic neural cells and/or progenitors thereof including, but not limited to, cerebral endothelial cells, neurons, dopamine neurons, motor neurons, interneurons, oligodendrocytes, microglial cells, Schwann cells, glial cells, cerebral endothelial progenitor cells, neuronal progenitors, dopamine neuronal progenitors, motor neuron progenitors, interneuron progenitors, oligodendrocyte progenitors, microglia. progenitors, Schwann progenitor cells, and glial progenitor cells described herein are administered to a subject to alleviate a symptom or effect of a spinal cord injury. In some embodiments, motor neurons, interneurons, Schwann cells, oligodendrocytes, microglia, and/or progenitors thereof are administered to a patient who has experienced a spinal cord injury.

In some embodiments, the hypoimmunogenic neural cells and/or progenitors thereof including, but not limited to, cerebral endothelial cells, neurons, dopamine neurons, motor neurons, interneurons, oligodendrocytes, microglial cells, Schwann cells, glial cells, cerebral endothelial progenitor cells, neuronal progenitors, dopamine neuronal progenitors, motor neuron progenitors, interneuron progenitors, oligodendrocyte progenitors, microglia. progenitors, Schwann progenitor cells, and glial progenitor cells described herein are administered to a subject to alleviate a symptom or effect of Pelizaeus-Merzbacher Disease. In some embodiments, oligodendrocytes and/or oligodendrocyte progenitors are administered to a subject with Pelizaeus-Merzbacher Disease.

In some embodiments, the hypoimmunogenic neural cells and/or progenitors thereof including, but not limited to, cerebral endothelial cells, neurons, dopamine neurons, motor neurons, interneurons, oligodendrocytes, microglial cells, Schwann cells, glial cells, cerebral endothelial progenitor cells, neuronal progenitors, dopamine neuronal progenitors, motor neuron progenitors, interneuron progenitors, oligodendrocyte progenitors, microglia. progenitors, Schwann progenitor cells, and glial progenitor cells described herein are administered to a subject to alleviate a symptom or effect of progressive multiple sclerosis. In some embodiments, the neural cells and/or neural progenitors are administered to a subject with progressive multiple sclerosis.

In some embodiments, the hypoimmunogenic neural cells and/or progenitors thereof including, but not limited to, cerebral endothelial cells, neurons, dopamine neurons, motor neurons, interneurons, oligodendrocytes, microglial cells, Schwann cells, glial cells, cerebral endothelial progenitor cells, neuronal progenitors, dopamine neuronal progenitors, motor neuron progenitors, interneuron progenitors, oligodendrocyte progenitors, microglia. progenitors, Schwann progenitor cells, and glial progenitor cells described herein are administered to a subject to alleviate a symptom or effect of Huntington’s Disease. In some embodiments, the neural cells and/or neural progenitors are administered to a subject Huntington’s Disease.

### B. Differentiation Methods to Generate Neural Cells

Provided herein are different neural cell types differentiated from hypoimmune pluripotent stem cells including hypoimmune induced pluripotent stem cells. The neural cell types are useful for subsequent transplantation or engraftment into subjects in need thereof. As will be appreciated by those in the art, the methods for differentiation depend on the desired cell type using known techniques.

In some embodiments, differentiation of pluripotent stem cells such as induced pluripotent stem cells is performed by exposing or contacting cells to specific factors which are known to produce a specific cell lineage(s), so as to target their differentiation to a specific, desired lineage and/or cell type of interest. In some embodiments, terminally differentiated neural cells display specialized phenotypic characteristics or features. In certain embodiments, the pluripotent stem cells are differentiated into neuroectodermal cells, neuronal cells, neuroendocrine cells, dopaminergic neurons, cholinergic neurons, serotonergic neurons, glutamatergic neurons, GABAergic neurons, adrenergic, noradrenergic neurons, sympathetic neurons, parasympathetic neurons, sympathetic peripheral neurons, glial cells, progenitors thereof, or precursors thereof. In some instances, the glial cells include microglial (e.g., amoeboid, ramified, activated phagocytic, and activated non-phagocytic) cells or macroglial cells (central nervous system cells: astrocytes, oligodendrocytes, ependymal cells, and radial glia; and peripheral nervous system cells: Schwann cells and satellite cells), precursors thereof, and progenitors of any of the preceding cells.

Protocols for generating different types of neural cells are described in PCT Application No. WO2010144696, U.S. Pat. Nos. 9,057,053; 9,376,664; and 10,233,422. Additional descriptions of methods for differentiating hypoimmunogenic pluripotent cells can be found, for example, in Deuse et al., Nature Biotechnology, 2019, 37, 252-258 and Han et al., Proc Natl Acad Sci USA, 2019, 116(21), 10441-10446.

### 1. Generating Cerebral Endothelial Cells

In some embodiments, cerebral endothelial cells (ECs), precursors, and progenitors thereof are differentiated from pluripotent stem cells (e.g., induced pluripotent stem cells) on a surface by culturing the cells in a medium comprising one or more factors that promote the generation of cerebral ECs or neural cell. In some instances, the medium includes one or more of the following: CHIR-99021, VEGF, basic FGF, and Y-27632. In some embodiments, the medium includes a supplement designed to promote survival and functionality for neural cells.

In some embodiments, cerebral endothelial cells (ECs), precursors, and progenitors thereof are differentiated from pluripotent stem cells on a surface by culturing the cells in an unconditioned or conditioned medium. In some instances, the medium comprises factors or small molecules that promote or facilitate differentiation. In some embodiments, the medium comprises one or more factors or small molecules selected from the group consisting of VEGFR, FGF, SDF-1, CHIR-99021, Y-27632, SB 431542, and any combination thereof. In some embodiments, the surface for differentiation comprises one or more extracellular matrix proteins. The surface can be coated with the one or more extracellular matrix proteins. The cells can be differentiated in suspension and then put into a gel matrix form, such as matrigel, gelatin, or fibrin/thrombin forms to facilitate cell survival. In some cases, differentiation is assayed as is known in the art, generally by evaluating the presence of cell-specific markers.

In some embodiments, the cerebral endothelial cells express or secrete a factor selected from the group consisting of CD31, VE cadherin, and a combination thereof. In certain embodiments, the cerebral endothelial cells express or secrete one or more of the factors selected from the group consisting of CD31, CD34, CD45, CD117 (c-kit), CD146, CXCR4, VEGF, SDF-1, PDGF, GLUT-1, PECAM-1, eNOS, claudin-5, occludin, ZO-1, p-glycoprotein, von Willebrand factor, VE-cadherin, low density lipoprotein receptor LDLR, low density lipoprotein receptor-related protein 1 LRP1, insulin receptor INSR, leptin receptor LEPR, basal cell adhesion molecule BCAM, transferrin receptor TFRC, advanced glycation endproduct-specific receptor AGER, receptor for retinol uptake STRA6, large neutral amino acids transporter small subunit 1 SLC7A5, excitatory amino acid transporter 3 SLC1A1, sodium-coupled neutral amino acid transporter 5 SLC38A5, solute carrier family 16 member 1 SLC16A1, ATP-dependent translocase ABCB1, ATP- ABCC2 binding cassette transporter ABCG2, multidrug resistance-associated protein 1 ABCC1, canalicular multispecific organic anion transporter 1 ABCC2, multidrug resistance-associated protein 4 ABCC4, and multidrug resistance-associated protein 5 ABCC5.

In some embodiments, the cerebral ECs are characterized with one or more of the features selected from the group consisting of high expression of tight junctions, high electrical resistance, low fenestration, small perivascular space, high prevalence of insulin and transferrin receptors, and high number of mitochondria.

In some embodiments, cerebral ECs are selected or purified using a positive selection strategy. In some instances, the cerebral ECs are sorted against an endothelial cell marker such as, but not limited to, CD31. In other words, CD31 positive cerebral ECs are isolated. In some embodiments, cerebral ECs are selected or purified using a negative selection strategy. In some embodiments, undifferentiated or pluripotent stem cells are removed by selecting for cells that express a pluripotency marker including, but not limited to, TRA-1-60 and SSEA-1.

### 2. Generating Neurons Including Dopamine Neurons

In some embodiments, hypoimmune cells described herein are dopamine neurons include neuronal stem cells, neuronal progenitor cells, immature dopamine neurons, and mature dopamine neurons that have been differentiated from pluripotent stem cells such as induced pluripotent stem cells.

Dopamine neurons (also referred to as dopaminergic neurons) include neuronal cells which express tyrosine hydroxylase (TH), the rate-limiting enzyme for dopamine synthesis. In some embodiments, dopamine neurons secrete the neurotransmitter dopamine, and have little or no expression of dopamine hydroxylase. A dopamine (DA) neuron can express one or more of the following markers: neuron-specific enolase (NSE), 1-aromatic amino acid decarboxylase, vesicular monoamine transporter 2, dopamine transporter, Nurrl, and dopamine-2 receptor (D2 receptor).

In some embodiments, hypoimmunogenic DA neurons, precursors, and progenitors thereof are differentiated from pluripotent stem cells by culturing the stem cells in medium comprising one or more factors or additives. Useful factors and additives that promote differentiation, growth, expansion, maintenance, and/or maturation of DA neurons include, but are not limited to, Wntl, FGF2, FGF8, FGF8a, sonic hedgehog (SHH), brain derived neurotrophic factor (BDNF), transforming growth factor a (TGF-a), TGF-b, interleukin 1 beta, glial cell line-derived neurotrophic factor (GDNF), a GSK-3 inhibitor (e.g., CHIR-99021), a TGF-b inhibitor (e.g., SB-431542), B-27 supplement, dorsomorphin, purmorphamine, noggin, retinoic acid, cAMP, ascorbic acid, neurturin, knockout serum replacement, N-acetyl cysteine, c-kit ligand, modified forms thereof, mimics thereof, analogs thereof, and variants thereof. In some embodiments, the DA neurons are differentiated in the presence of one or more factors that activate or inhibit the WNT pathway, NOTCH pathway, SHH pathway, BMP pathway, FGF pathway, and the like.

Differentiation protocols to generate DA neurons and progenitors thereof and detailed descriptions of such are provided in, e.g., WO2020/018615; U.S. Pat. Nos. 9,968,637 and 7,674,620; Kim et al, Nature, 2002, 418,50-56; Bjorklund et al, PNAS, 2002, 99(4), 2344-2349; Grow et al., Stem Cells Transl Med. 2016, 5(9): 1133-44, and Cho et al, PNAS, 2008, 105:3392-3397, the disclosures in their entirety including the detailed description of the examples, methods, figures, and results are herein incorporated by reference.

In some embodiments, a population of hypoimmunogenic DA neurons is isolated from non-neuronal cells. In some embodiments, the isolated population of hypoimmunogenic DA neurons is expanded prior to administration. In certain embodiments, the isolated population of hypoimmunogenic DA neurons is expanded and cryopreserved prior to administration.

To characterize and monitor DA differentiation and assess the DA phenotype, expression of any number of molecular and genetic markers can be evaluated. For example, the presence of genetic markers can be determined by various methods known to those skilled in the art. Expression of molecular markers can be determined by quantifying methods such as, but not limited to, qPCR-based assays, immunoassays, immunocytochemistry assays, immunoblotting assays, and the like. Exemplary markers for DA neurons include, but are not limited to, TH, beta-tubulin, paired box protein (Pax6), insulin gene enhancer protein (Isl1), nestin, diaminobenzidine (DAB), G protein-activated inward rectifier potassium channel 2 (GIRK2), microtubule-associated protein 2 (MAP-2), Nurrl, dopamine transporter (DAT), forkhead box protein A2 (FOXA2), FOX3, doublecortin, and LIM homeobox transcription factor 1-beta (LMX1B), and the like. In some embodiments, the DA neurons express one or more of the markers selected from corin, FOXA2, TuJ1, NURR1, and any combination thereof.

In some embodiments, DA neurons are assessed according to cell electrophysiological activity. The electrophysiology of the cells can be evaluated by using assays knowns to those skilled in the art. For instance, whole-cell and perforated patch clamp, assays for detecting electrophysiological activity of cells, assays for measuring the magnitude and duration of action potential of cells, and functional assays for detecting dopamine production of DA cells.

In some embodiments, DA neuron differentiation is characterized by spontaneous rhythmic action potentials, and high-frequency action potentials with spike frequency adaption upon injection of depolarizing current. In other embodiments, DA differentiation is characterized by the production of dopamine. The level of dopamine produced is calculated by measuring the width of an action potential at the point at which it has reached half of its maximum amplitude (spike half-maximal width).

In some embodiments, the hypoimmunogenic DA neurons are administered to a patient, e.g., human patient to treat a neurodegenerative disease or condition. In some cases, the neurodegenerative disease or condition is selected from the group consisting of Parkinson’s disease, Huntington disease, and multiple sclerosis. In other embodiments, the DA neurons are used to treat or ameliorate one or more symptoms of a neuropsychiatric disorder, such as attention deficit hyperactivity disorder (ADHD), Tourette Syndrome (TS), schizophrenia, psychosis, and depression. In yet other embodiments, the DA neurons are used to treat a patient with impaired DA neurons.

In some embodiments, the differentiated DA neurons are transplanted either intravenously or by injection at particular locations in the patient. In some embodiments, the differentiated DA cells are transplanted into the substantia nigra (particularly in or adjacent of the compact region), the ventral tegmental area (VTA), the caudate, the putamen, the nucleus accumbens, the subthalamic nucleus, or any combination thereof, of the brain to replace the DA neurons whose degeneration resulted in Parkinson’s disease. The differentiated DA cells can be injected into the target area as a cell suspension. Alternatively, the differentiated DA cells can be embedded in a support matrix or scaffold when contained in such a delivery device. In some embodiments, the scaffold is biodegradable. In other embodiments, the scaffold is not biodegradable. The scaffold can comprise natural or synthetic (artificial) materials.

The delivery of the DA neurons can be achieved by using a suitable vehicle such as, but not limited to, liposomes, microparticles, or microcapsules. In other embodiments, the differentiated DA neurons are administered in a pharmaceutical composition comprising an isotonic excipient. The pharmaceutical composition is prepared under conditions that are sufficiently sterile for human administration. In some embodiments, the DA neurons differentiated from hypoimmunogenic iPSCs are supplied in the form of a pharmaceutical composition. General principles of therapeutic formulations of cell compositions are found in Cell Therapy: Stem Cell Transplantation, Gene Therapy, and Cellular Immunotherapy, G. Morstyn & W. Sheridan eds, Cambridge University Press, 1996, and Hematopoietic Stem Cell Therapy, E. Ball, J. Lister & P. Law, Churchill Livingstone, 2000, the disclosures are incorporated herein by reference.

Useful descriptions of various neuronal cell types derived from stem cells and methods of making thereof can be found, for example, in Kirkeby et al., Cell Rep, 2012, 1:703-714; Kriks et al., Nature, 2011, 480:547-551; Doi et al., Stem Cell Reports, 2014, 2, 337-50; Perrier et al., Proc Natl Acad Sci USA, 2004, 101, 12543-12548; Chambers et al., Nat Biotechnol, 2009, 27, 275-280; Wang et al., Stem Cell Reports, 2018, 11(1):171-182; Lorenz Studer, “Chapter 8 -Strategies for Bringing Stem Cell-Derived Dopamine Neurons to the clinic-The NYSTEM Trial” in Progress in Brain Research, 2017, volume 230, pg. 191-212; Liu et al., Nat Protoc, 2013, 8:1670-1679; Upadhya et al., Curr Protoc Stem Cell Biol, 38, 2D.7.1-2D.7.47; U.S. Publication Appl. No. 20160115448, and U.S. Pat. Nos. 8,252,586; 8,273,570; 9,487,752 and 10,093,897, the contents are incorporated herein by reference in their entirety.

In addition to DA neurons, other neurons, precursors, and progenitors thereof are differentiated from pluripotent stem cells by culturing the cells in medium comprising one or more factors selected from the group consisting of GDNF, BDNF, GM-CSF, B27, basic FGF, basic EGF, NGF, CNTF, SMAD inhibitor, Wnt antagonist, SHH signaling activator, and any combination thereof. In some embodiments, the SMAD inhibitor is selected from the group consisting of SB431542, LDN-193189, Noggin PD169316, SB203580, LY364947, A77-01, A-83-01, BMP4, GW788388, GW6604, SB-505124, lerdelimumab, metelimumab, GC-I008, AP-12009, AP-110I4, LY550410, LY580276, LY364947, LY2109761, SB-505124, E-616452 (RepSox ALK inhibitor), SD-208, SMI6, NPC-30345, K 26894, SB-203580, SD-093, activin-M108A, P144, soluble TBR2-Fc, DMH-1, dorsomorphin dihydrochloride and derivatives thereof. In some embodiments, the Wnt antagonist is selected from the group consisting of XAV939, DKK1, DKK-2, DKK-3, Dkk-4, SFRP-1, SFRP-2, SFRP-5, SFRP-3, SFRP-4, WIF-1, Soggy, IWP-2, IWR1, ICG-001, KY0211, Wnt-059, LGK974, IWP-L6 and derivatives thereof. In some embodiments, the SHH signaling activator is selected from the group consisting of Smoothened agonist (SAG), SAG analog, SHH, C25-SHH, C24-SHH, purmorphamine, Hg--Ag and derivatives thereof. In some embodiments, the differentiation medium contains a supplement or additive to induce neuronal differentiation. In some embodiments, the cells are cultured in the presence of a supplement or additive to induce floor plate cells. In some embodiments, the supplement or additive includes BMP inhibitor LDN193189, ALK-5 inhibitor A83-01, Smoothened agonist purmorphamine, FGF8, GSK3 inhibitor CHIR99021, glial cell line-derived neurotrophic factor, GDNF, ascorbic acid, brain-derived neurotrophic factor BDNF, dibutyryladenosine cyclic monophosphate dbcAMP, ROCK inhibitor Y-27632, and the like.

In some embodiments, the neurons and progenitors described herein express one or more of the markers selected from the group consisting of glutamate ionotropic receptor NMDA type subunit 1 GRIN1, glutamate decarboxylase 1 GAD1, gamma-aminobutyric acid GABA, tyrosine hydroxylase TH, LIM homeobox transcription factor 1-alpha LMX1A, Forkhead box protein O1 FOXO1, Forkhead box protein A2 FOXA2, Forkhead box protein 04 FOXO4, FOXG1, 2′,3′-cyclic-nucleotide 3′-phosphodiesterase CNP, myelin basic protein MBP, tubulin beta chain 3 TUB3, tubulin beta chain 3 NEUN, solute carrier family 1 member 6 SLC1A6, SST, PV, calbindin, RAX, LHX6, LHX8, DLX1, DLX2, DLX5, DLX6, SOX6, MAFB, NPAS1, ASCL1, SIX6, OLIG2, NKX2.1, NKX2.2, NKX6.2, VGLUT1, MAP2, CTIP2, SATB2, TBR1, DLX2, ASCL1, ChAT, NGFI-B, c-fos, CRF, RAX, POMC, hypocretin, NADPH, NGF, Ach, VAChT, PAX6, EMX2p75, and any combination thereof.

### 3. Generating Glial Cells

In some embodiments, the neural cells described include glial cells such as, but not limited to, microglia, astrocytes, oligodendrocytes, ependymal cells and Schwann cells, glial precursors, and glial progenitors thereof are produced by differentiating pluripotent stem cells into therapeutically effective glial cells and the like.

In some embodiments, glial cells, precursors, and progenitors thereof are generated by culturing pluripotent stem cells in medium comprising one or more agents selected from the group consisting of retinoic acid, IL-34, M-CSF, FLT3 ligand, GM-CSF, CCL2, a TGFbeta inhibitor, a BMP signaling inhibitor, a SHH signaling activator, FGF, platelet derived growth factor PDGF, PDGFR-alpha, HGF, IGF-1, noggin, sonic hedgehog (SHH), dorsomorphin, noggin, and any combination thereof. In certain instances, the BMP signaling inhibitor is LDN193189, SB431542, or a combination thereof. Exemplary differentiation medium can include any specific factors and/or small molecules that may facilitate or enable the generation of a glial cell type as recognized by those skilled in the art.

In some embodiments, differentiation of pluripotent stem cells is performed by exposing or contacting cells to specific factors which are known to produce a glial cell such as a microglial cell (such as a amoeboid, ramified, activated phagocytic, and activated non-phagocytic cell), a macroglial cell (such as a astrocyte, oligodendrocyte, ependymal cell, radial glia, Schwann cell and satellite cell, a precursor thereof, and a progenitor thereof. Useful methods for generating glial cells, precursors, and progenitors thereof from stem cells are found, for example, in U.S. Pat. Nos. 7,579,188; 7,595,194; 8,263,402; 8,206,699; 8,227,247; 8,252,586; 9,193,951; 9,709,553; and 9,862,925; and US Publ. Application Nos. 2018/0187148; 2017/0198255; 2017/0183627; 2017/0182097; 2017/253856; 2018/0236004; and PCT Publ. Application Nos. WO2017/172976 and WO2018/093681.

In some embodiments, the glial cells express NKX2.2, PAX6, SOX10, brain derived neurotrophic factor BDNF, neutrotrophin-3 NT-3, NT-4, epidermal growth factor EGF, ciliary neurotrophic factor CNTF, nerve growth factor NGF, FGF8, EGFR, OLIG1, OLIG2, myelin basic protein MBP, GAP-43, LNGFR, nestin, GFAP, CD11b, CD11c, CD105, CX3CR1, P2RY12, IBA-1, TMEM119, CD45, and any combination thereof. Any of these exemplary markers can be used to characterize glial cells described herein. To monitor glial cell differentiation as well as to assess the phenotype of a glial cell, the expression of any number of molecular and genetic markers specific to glial cells and progenitors thereof can be evaluated. For example, the presence of genetic markers can be determined by various methods known to those skilled in the art. Expression of molecular markers can be determined by quantifying methods such as, but not limited to, qPCR-based assays, RNA-seq assays, proteomic assays, immunoassays, immunocytochemistry assays, immunoblotting assays, and the like.

In some embodiments, the glial cells including oligodendrocytes, astrocytes, and progenitors thereof express one or more of the markers selected from A2B5, CD9, CD133, CD140a, FOXG1, GalC, GD3, GFAP, nestin, NG2, MBP, Musashi, 04, Olig1, Olig2, PDGFαR, S100β, glutamine synthetase, connexin 43, vimentin, BLBP, GLAST, and the like. In some embodiments, the glial cells including oligodendrocytes, astrocytes, and progenitors thereof do not express one or more of the markers selected from PSA-NCAM, CD9, CD11, CD32, CD36, CD105, CD140a, nestin, PDGFαR, and the like. In some embodiments, the glial cells are selected or purified using a positive selection strategy, a negative selection strategy, or both.

In some embodiments, glial cells are characterized according to morphology as determined by immunocytochemistry and immunohistochemistry. In some embodiments, glial cells are assessed according to functional characterization assays such as, but not limited to, a neuronal co-culture assay, stimulation assay with lipopolysaccharides (LPS), in vitro myelination assay, ATP influx with calcium wave oscillation assay, and the like.

In some embodiments, to determine that the glial cells display cell-specific characteristics and features, the cells can be transplanted into an animal model. In some embodiments, the glial cells are injected into an immunocompromised mouse, e.g., an immunocompromised shiverer mouse. The glial cells are administered to the brain of the mouse and after a pre-selected amount of time, the engrafted cells are evaluated. In some instances, the engrafted cells in the brain are visualized using immunostaining and imaging methods. In some embodiments, expression of known glial cell biomarkers can be determined in the engrafted cells.

Additional methods for determining the effect of neural cell transplantation in an animal model of a neurological disorder or condition are described in the following references: for spinal cord injury - Curtis et al., Cell Stem Cell, 2018, 22, 941-950; for Parkinson’s disease - Kikuchi et al., Nature, 2017, 548:592-596; for ALS - Izrael et al., Stem Cell Research, 2018, 9(1): 152 and Izrael et al., IntechOpen, DOI: 10.5772/intechopen.72862; for epilepsy - Upadhya et al., PNAS, 2019, 116(1):287-296.

The efficacy of neural cell transplants for spinal cord injury can be assessed in, for example, a rat model for acutely injured spinal cord, as described by McDonald et al., Nat. Med., 1999, 5:1410 and Kim et al., Nature, 2002, 418:50. For instance, successful transplants may show transplant-derived cells present in the lesion 2-5 weeks later, differentiated into astrocytes, oligodendrocytes, and/or neurons, and migrating along the spinal cord from the lesioned end, and an improvement in gait, coordination, and weight-bearing. Specific animal models are selected based on the neural cell type and neurological disease or condition to be treated.

### C. Hypoimmunogenic Cells

The present technology is directed to neural cells derived from pluripotent stem cells (e.g., pluripotent stem cells and induced pluripotent stem cells) that overexpress CD24 or CD47. The neural cells overexpress CD24 or CD47 and evade immune recognition. In some embodiments, the neural cells and pluripotent stem cells display reduced levels or activity of MHC class I antigens and/or MHC class II antigens.

Methods provided are useful for inactivation or ablation of MHC class I expression and/or MHC class II expression in cells such as but not limited to pluripotent stem cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of critical immune genes (e.g., by deleting genomic DNA of critical immune genes) in human stem cells. In certain embodiments, genome editing technologies or other gene modulation technologies are used to insert tolerance-inducing factors in human cells, rendering them and the differentiated cells prepared therefrom hypoimmunogenic cells. As such, the hypoimmunogenic cells have reduced or eliminated expression of MHC I and MHC II expression. In some embodiments, the cells are nonimmunogenic (e.g., do not induce an immune response) in a recipient subject.

The genome editing techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

The practice of the some embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

Provided herein are cells comprising a modification of one or more targeted polynucleotide sequences that regulates the expression of MHC I and/or MHC II. In certain embodiments, the modification comprising increasing expression of CD47. In some embodiments, the cells comprise an exogenous or recombinant CD47 polypeptide. Also, provided herein are cells comprising a modification of one or more targeted polynucleotide sequences that regulates the expression of MHC I and/or MHC II. In certain embodiments, the modification comprising increasing expression of CD24. In some embodiments, the cells comprise an exogenous CD24 or recombinant polypeptide. In some embodiments, the cell also includes a modification to increase expression of one selected from the group consisting of CD200, HLA-G, HLA-E, HLA-C, HLA-E heavy chain, PD-L1, IDO1, CTLA4-Ig, IL-10, IL-35, FASL, Serpinb9, CCL21, CCL22, and Mfge8. In some embodiments, the cell further comprises a tolerogenic factor (e.g., an immunomodulatory molecule) selected from the group consisting of DUX4, CD200, HLA-G, HLA-E, HLA-C, HLA-E heavy chain, PD-L1, IDO1, CTLA4-Ig, IL-10, IL-35, FASL, Serpinb9, CCL21, CCL22, and Mfge8.

In some embodiments, the cell comprises a genomic modification of one or more targeted polynucleotide sequences that regulates the expression of MHC I and/or MHC II. In some embodiments, a genetic editing system is used to modify one or more targeted polynucleotide sequences. In some embodiments, the targeted polynucleotide sequence is one or more selected from the group consisting of B2M and CIITA. In some cases, the targeted polynucleotide sequence is NLRC5. In certain embodiments, the genome of the cell has been altered to reduce or delete critical components of HLA expression.

In some embodiments, the present disclosure provides a stem cell (e.g., pluripotent stem cell or induced pluripotent stem cell) or population thereof comprising a genome in which a gene has been edited to delete a contiguous stretch of genomic DNA, thereby reducing or eliminating surface expression of MHC class I molecules in the cell or population thereof. In certain embodiments, the present disclosure provides a stem cell (e.g., pluripotent stem cell or induced pluripotent stem cell) or population thereof comprising a genome in which a gene has been edited to delete a contiguous stretch of genomic DNA, thereby reducing or eliminating surface expression of MHC class II molecules in the cell or population thereof. In some embodiments, the present disclosure provides a stem cell (e.g., pluripotent stem cell or induced pluripotent stem cell) or population thereof comprising a genome in which one or more genes has been edited to delete a contiguous stretch of genomic DNA, thereby reducing or eliminating surface expression of MHC class I and II molecules in the cell or population thereof.

In certain embodiments, the expression of MHC I or MHC II is modulated by targeting and deleting a contiguous stretch of genomic DNA thereby reducing or eliminating expression of a target gene selected from the group consisting of B2M and CIITA. In other cases, the target gene is NLRC5.

In some embodiments, the cells and methods described herein include genomically editing human cells to cleave CIITA gene sequences as well as editing the genome of such cells to alter one or more additional target polynucleotide sequences such as, but not limited to, B2M and NLRC5. In some embodiments, the cells and methods described herein include genomically editing human cells to cleave B2M gene sequences as well as editing the genome of such cells to alter one or more additional target polynucleotide sequences such as, but not limited to, CIITA and NLRC5. In some embodiments, the cells and methods described herein include genomically editing human cells to cleave NLRC5 gene sequences as well as editing the genome of such cells to alter one or more additional target polynucleotide sequences such as, but not limited to, B2M and CIITA.

### D. CD47

In some embodiments, the present disclosure provides a stem cell (e.g., pluripotent stem cell or induced pluripotent stem cell) or population thereof that has been modified to express the tolerogenic factor (e.g., immunomodulatory polypeptide) CD47. In some embodiments, the present disclosure provides a method for altering a stem cell genome to express CD47. In some embodiments, the stem cell expresses exogenous CD47. In some instances, the stem cell expresses an expression vector comprising a nucleotide sequence encoding a human CD47 polypeptide.

CD47 is a leukocyte surface antigen and has a role in cell adhesion and modulation of integrins. It is expressed on the surface of a cell and signals to circulating macrophages not to eat the cell.

In some embodiments, the cell of the present technology comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell comprises a nucleotide sequence encoding a CD47 polypeptide having an amino acid sequence as set forth in NCBI Ref. Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Sequence Nos. NM_001777.3 and NM_198793.2. In some embodiments, the cell comprises a nucleotide sequence for CD47 as set forth in NCBI Ref. Sequence Nos. NM_001777.3 and NM_198793.2.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell in accordance with the present technology comprises a CD47 polypeptide having an amino acid sequence as set forth in NCBI Ref. Sequence Nos. NP_001768.1 and NP_942088.1.

In another embodiment, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

### E. CD24

In some embodiments, the present disclosure provides a stem cell (e.g., pluripotent stem cell or induced pluripotent stem cell) or population thereof that has been modified to express the tolerogenic factor (e.g., immunomodulatory polypeptide) CD24. In some embodiments, the present disclosure provides a method for altering a stem cell genome to express CD24. In some embodiments, the stem cell expresses exogenous CD24. In some instances, the stem cell expresses an expression vector comprising a nucleotide sequence encoding a human CD24 polypeptide.

CD24 which is also referred to as a heat stable antigen or small-cell lung cancer cluster 4 antigen is a glycosylated glycosylphosphatidylinositol-anchored surface protein (Pirruccello et al., J Immunol, 1986, 136, 3779-3784; Chen et al., Glycobiology, 2017, 57, 800-806). It binds to Siglec-10 on innate immune cells. Recently it has been shown that CD24 via Siglec-10 acts as an innate immune checkpoint (Barkal et al., Nature, 2019, 572, 392-396).

In some embodiments, the cell in accordance with the present technology comprises a nucleotide sequence encoding a CD24 polypeptide has at least 95% sequence identity (e.g., 95%, 96%, 97%, 98%, 99%, or more) to an amino acid sequence set forth in NCBI Ref. Nos. NP_001278666.1, NP_001278667.1, NP _001278668.1, and NP_037362.1. In some embodiments, the cell comprises a nucleotide sequence encoding a CD24 polypeptide having an amino acid sequence set forth in NCBI Ref. Nos. NP_001278666.1, NP_001278667.1, NP_001278668.1, and NP_037362.1.

In some embodiments, the cell comprises a nucleotide sequence having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_00129737.1, NM_00129738.1, NM_001291739.1, and NM_013230.3. In some embodiments, the cell comprises a nucleotide sequence as set forth in NCBI Ref. Nos. NM_00129737.1, NM_00129738.1, NM_001291739.1, and NM_013230.3.

In another embodiment, CD24 protein expression is detected using a Western blot of cells lysates probed with antibodies against the CD24 protein. In another embodiment, reverse transcriptase polymerase chain reactions (RT-PCR) are used to confirm the presence of the exogenous CD24 mRNA.

### F. CIITA

In certain embodiments, the present technology modulates (e.g., reduces or eliminates) the expression of MHC II genes by targeting and modulating (e.g., reducing or eliminating) Class II transactivator (CIITA) expression. In some embodiments, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the hypoimmunogenic cells in accordance with the present technology comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS:5184-36352 of Table 12 of WO2016183041, which is herein incorporated by reference. In some embodiments, the cell has a reduced ability to induce an immune response in a recipient subject.

Assays to test whether the CIITA gene has been inactivated are known and described herein. In one embodiment, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification.

### G. B2M

In certain embodiments, the present technology modulates (e.g., reduces or eliminates) the expression of MHC-I genes by targeting and modulating (e.g., reducing or eliminating) expression of the accessory chain B2M. In some embodiments, the modulation occurs using a CRISPR/Cas system. By modulating (e.g., reducing or deleting) expression of B2M, surface trafficking of MHC-I molecules is blocked and the cell rendered hypoimmunogenic. In some embodiments, the cell has a reduced ability to induce an immune response in a recipient subject.

In some embodiments, the target polynucleotide sequence is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules - HLA-A, HLA-B, and HLA-C.

In some embodiments, the hypoimmunogenic cells in accordance with the present technology comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS:81240-85644 of Table 15 of WO2016/183041, which is herein incorporated by reference.

Assays to test whether the B2M gene has been inactivated are known and described herein. In one embodiment, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

### H. Additional Tolerogenic Factors

In certain embodiments, one or more tolerogenic factors can be inserted or reinserted into genome-edited cells to create immune-privileged universal donor cells, such as universal donor stem cells. In certain embodiments, the hypoimmunogenic cells (e.g., hypoimmunogenic stem cells) disclosed herein have been further modified to express one or more tolerogenic factors. Exemplary tolerogenic factors include, without limitation, one or more of DUX4, CD200, HLA-G, HLA-E, HLA-C, HLA-E heavy chain, PD-L1, IDO1, CTLA4-Ig, IL-10, IL-35, FASL, Serpinb9, CCL21, CCL22, and Mfge8. In some embodiments, the tolerogenic factors are selected from the group consisting of CD200, HLA-G, HLA-E, HLA-C, HLA-E heavy chain, PD-L1, IDO1, CTLA4-Ig, IL-10, IL-35, FASL, Serpinb9, CCL21, CCL22, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G, PD-L1, CTLA-4-Ig, C1-inhibitor, and IL-35.

In some instances, a gene editing system such as the CRISPR/Cas system is used to facilitate the insertion of tolerogenic factors, such as the tolerogenic factors into a safe harbor locus, such as the AAVS 1 locus, to actively inhibit immune rejection. In some instances, the tolerogenic factors are inserted into a safe harbor locus using an expression vector.

In some embodiments, the present disclosure provides a stem cell (e.g., hypoimmunogenic stem cell such as a hypoimmunogenic pluripotent stem cell or a hypoimmunogenic iPSC) or population thereof comprising a genome in which the stem cell genome has been modified to express CD47. In some embodiments, the present disclosure provides a method for altering a stem cell genome to express CD47. In certain embodiments at least one ribonucleic acid or at least one pair of ribonucleic acids may be utilized to facilitate the insertion of CD47 into a stem cell line. In certain embodiments, the at least one ribonucleic acid or the at least one pair of ribonucleic acids is selected from the group consisting of SEQ ID NOS:200784-231885 of Table 29 of WO2016183041, which is herein incorporated by reference.

In some embodiments, the present disclosure provides a stem cell (e.g., hypoimmunogenic stem cell such as a hypoimmunogenic pluripotent stem cell or a hypoimmunogenic iPSC) or population thereof comprising a genome in which the stem cell genome has been modified to express HLA-C. In some embodiments, the present disclosure provides a method for altering a stem cell genome to express HLA-C. In certain embodiments, at least one ribonucleic acid or at least one pair of ribonucleic acids may be utilized to facilitate the insertion of HLA-C into a stem cell line. In certain embodiments, the at least one ribonucleic acid or the at least one pair of ribonucleic acids is selected from the group consisting of SEQ ID NOS:3278-5183 of Table 10 of WO2016183041, which is herein incorporated by reference.

In some embodiments, the present disclosure provides a stem cell (e.g., hypoimmunogenic stem cell such as a hypoimmunogenic pluripotent stem cell or a hypoimmunogenic iPSC) or population thereof comprising a genome in which the stem cell genome has been modified to express HLA-E. In some embodiments, the present disclosure provides a method for altering a stem cell genome to express HLA-E. In certain embodiments, at least one ribonucleic acid or at least one pair of ribonucleic acids may be utilized to facilitate the insertion of HLA-E into a stem cell line. In certain embodiments, the at least one ribonucleic acid or the at least one pair of ribonucleic acids is selected from the group consisting of SEQ ID NOS:189859-193183 of Table 19 of WO2016183041, which is herein incorporated by reference.

In some embodiments, the present disclosure provides a stem cell (e.g., hypoimmunogenic stem cell such as a hypoimmunogenic pluripotent stem cell or a hypoimmunogenic iPSC) or population thereof comprising a genome in which the stem cell genome has been modified to express HLA-F. In some embodiments, the present disclosure provides a method for altering a stem cell genome to express HLA-F. In certain embodiments, at least one ribonucleic acid or at least one pair of ribonucleic acids may be utilized to facilitate the insertion of HLA-F into a stem cell line. In certain embodiments, the at least one ribonucleic acid or the at least one pair of ribonucleic acids is selected from the group consisting of SEQ ID NOS: 688808-399754 of Table 45 of WO2016183041, which is herein incorporated by reference.

In some embodiments, the present disclosure provides a stem cell (e.g., hypoimmunogenic stem cell such as a hypoimmunogenic pluripotent stem cell or a hypoimmunogenic iPSC) or population thereof comprising a genome in which the stem cell genome has been modified to express HLA-G. In some embodiments, the present disclosure provides a method for altering a stem cell genome to express HLA-G. In certain embodiments at least one ribonucleic acid or at least one pair of ribonucleic acids may be utilized to facilitate the insertion of HLA-G into a stem cell line. In certain embodiments, the at least one ribonucleic acid or the at least one pair of ribonucleic acids is selected from the group consisting of SEQ ID NOS:188372-189858 of Table 18 of WO2016183041, which is herein incorporated by reference.

In some embodiments, the present disclosure provides a stem cell (e.g., hypoimmunogenic stem cell such as a hypoimmunogenic pluripotent stem cell or a hypoimmunogenic iPSC) or population thereof comprising a genome in which the stem cell genome has been modified to express PD-L1. In some embodiments, the present disclosure provides a method for altering a stem cell genome to express PD-L1. In certain embodiments, at least one ribonucleic acid or at least one pair of ribonucleic acids may be utilized to facilitate the insertion of PD-L1 into a stem cell line. In certain embodiments, the at least one ribonucleic acid or the at least one pair of ribonucleic acids is selected from the group consisting of SEQ ID NOS:193184-200783 of Table 21 of WO2016183041, which is herein incorporated by reference.

In some embodiments, the present disclosure provides a stem cell (e.g., hypoimmunogenic stem cell such as a hypoimmunogenic pluripotent stem cell or a hypoimmunogenic iPSC) or population thereof comprising a genome in which the stem cell genome has been modified to express CTLA4-Ig. In some embodiments, the present disclosure provides a method for altering a stem cell genome to express CTLA4-Ig. In certain embodiments, at least one ribonucleic acid or at least one pair of ribonucleic acids may be utilized to facilitate the insertion of CTLA4-Ig into a stem cell line. In certain embodiments, the at least one ribonucleic acid or the at least one pair of ribonucleic acids is selected from any one disclosed in WO2016183041, including the sequence listing.

In some embodiments, the present disclosure provides a stem cell (e.g., hypoimmunogenic stem cell) or population thereof comprising a genome in which the stem cell genome has been modified to express CI-inhibitor. In some embodiments, the present disclosure provides a method for altering a stem cell genome to express CI-inhibitor. In certain embodiments, at least one ribonucleic acid or at least one pair of ribonucleic acids may be utilized to facilitate the insertion of CI-inhibitor into a stem cell line. In certain embodiments, the at least one ribonucleic acid or the at least one pair of ribonucleic acids is selected from any one disclosed in WO2016183041, including the sequence listing.

In some embodiments, the present disclosure provides a stem cell (e.g., hypoimmunogenic stem cell such as a hypoimmunogenic pluripotent stem cell or a hypoimmunogenic iPSC) or population thereof comprising a genome in which the stem cell genome has been modified to express IL-35. In some embodiments, the present disclosure provides a method for altering a stem cell genome to express IL-35. In certain embodiments, at least one ribonucleic acid or at least one pair of ribonucleic acids may be utilized to facilitate the insertion of IL-35 into a stem cell line. In certain embodiments, the at least one ribonucleic acid or the at least one pair of ribonucleic acids is selected from any one disclosed in WO2016183041, including the sequence listing.

In some embodiments, the tolerogenic factors are expressed in a cell using an expression vector. For example, the expression vector for expressing CD47 in a cell comprises a polynucleotide sequence encoding CD47. The expression vector can be an inducible expression vector. The expression vector can be a viral vector, such as but not limited to, a lentiviral vector.

In some embodiments, the present disclosure provides a stem cell (e.g., hypoimmunogenic stem cell such as a hypoimmunogenic pluripotent stem cell or a hypoimmunogenic iPSC) or population thereof comprising a genome in which the stem cell genome has been modified to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In some embodiments, the present disclosure provides a method for altering a stem cell genome to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDO1. In certain embodiments, at least one ribonucleic acid or at least one pair of ribonucleic acids may be utilized to facilitate the insertion of the selected polypeptide into a stem cell line. In certain embodiments, the at least one ribonucleic acid or the at least one pair of ribonucleic acids is selected from any one disclosed in Appendices 1-47 and the sequence listing of WO2016183041, the disclosure is incorporated herein by references.

### I. Methods of Genetic Modifications

In some embodiments, the rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

Target polynucleotide sequences described herein may be altered in any manner which is available to the skilled artisan utilizing a CRISPR/Cas system. Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, the CRISPR/Cas systems can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, a CRISPR/Cas system includes a Cas protein and at least one to two ribonucleic acids that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to, Cas1, Cas2, Cas3, Cas4, Cas5, Cas6, Cas7, Cas8 and Cas9. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019).

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods described contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA sequences useful for CRISPR/Cas-based targeting of genes described herein are provided in Table 1. The sequences can be found in WO2016/183041 filed May 9, 2016, the disclosure including the Tables, Appendices, and Sequence Listing is incorporated herein by reference in its entirety.

TABLE 1

EXEMPLARY GRNA SEQUENCES USEFUL FOR TARGETING GENES

| | | |
|---|---|---|
| Gene Name | SEQ ID NO: (WO2016183041) | WO2016183041 |
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOS:3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOS:5184-36352 | Table 12, Appendix 5 |
| B2M | SEQ ID NOS:81240-85644 | Table 15, Appendix 8 |
| NLRC5 | SEQ ID NOS:36353-81239 | Table 14, Appendix 7 |
| CD47 | SEQ ID NOS:200784-231885 | Table 29, Appendix 22 |
| HLA-E | SEQ ID NOS:189859-193183 | Table 19, Appendix 12 |
| HLA-F | SEQ ID NOS:688808-699754 | Table 45, Appendix 38 |
| HLA-G | SEQ ID NOS:188372-189858 | Table 18, Appendix 11 |
| PD-L1 | SEQ ID NOS: 193184-200783 | Table 21, Appendix 14 |

In some embodiments, the cells are made using Transcription Activator-Like Effector Nucleases (TALEN) methodologies.

By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues co-ordinated with zinc along with the two cysteine residues of a single beta turn (see, e.g., Berg & Shi, Science 271:1081-1085 (1996)).

In some embodiments, the cells are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. In some embodiments, the homing endonuclease may, for example, correspond to a LAGLIDADG endonuclease, to a HNH endonuclease, or to a GIY-YIG endonuclease. Preferred homing endonuclease can be an I-CreI variant.

In some embodiments, the cells are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells are made using RNA silencing or RNA interference (RNAi) to knockdown (e.g., decrease, eliminate, or inhibit) the expression of a polypeptide such as a tolerogenic factor. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, CIITA can be knocked down in a pluripotent stem cell by introducing a CIITA siRNA or transducing a CIITA shRNA-expressing virus into the cell. In some embodiments, RNA interference is employed to reduce or inhibit the expression of at least one selected from the group consisting of CIITA, B2M, and NLRC5.

### J. Overexpression of Tolerogenic Factors

For all of these technologies, well known recombinant techniques are used, to generate recombinant nucleic acids. In certain embodiments, the recombinant nucleic acids encoding a tolerogenic factor may be operably linked to one or more regulatory nucleotide sequences in an expression construct. Regulatory nucleotide sequences will generally be appropriate for the host cell and subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector’s copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al, Nature 273: 113-120 (1978)). The immediate early promoter of the human cytomegalovirus is conveniently obtained as a HindIIIE restriction fragment (Greenaway et al, Gene 18: 355-360 (1982)). Some other examples include the human β-actin promoter (ACTB) promoter, the cytomegalovirus (CMV) promoter, the elongation factor-1α (EF1α) promoter, the phosphoglycerate kinase (PGK) promoter, the synthetic CAG promoter, and the ubiquitinC (UbC) promoter. Additional promoters that may be used can be found in Norrman K et al., PLoS ONE, 2010, 5(8): e12413; Xia, Stem Cells Dev., 2007; 16(1): 167-176), the references herewith are incorporated by reference in their entirety.

In some embodiments, a tolerogenic factor such as CD47 expressed under the control of a constitutive promoter or a cell-specific promoter. In some cases, a constitutive promoter is selected from the group consisting of a CMV promoter, CAG promoter, EF1α promoter, PGK promoter, and ACTB promoter. In some cases, a cell-specific promoter is active in any of the neural cell types described herein. In some embodiments, exogenous CD47 expression in the cells of the present technology is controlled a cell-specific promoter that is active in a neural cell and/or a neural progenitor cell. In certain embodiments, the cell-specific promoter is active in a dopamine neuron and/or a progenitor thereof. In some cases, a cell-specific promoter is selected from the group consisting of TUJ1, MAP2, Synapsin, ENO2, TUBA1A, AADC, TH, VMAT2, DAT, NURR1, and PITX3.

In some embodiments, expression of a target gene (e.g., CD47, or another tolerogenic factor) is increased by expression of fusion protein or a protein complex containing (1) a site-specific binding domain specific for the endogenous CD47, or other gene and (2) a transcriptional activator.

In some embodiments, the regulatory factor is comprised of a site-specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs).

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site-specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. See also U.S. Pat. No. 5,420,032; U.S. Pat. No. 6,833,252; Belfort et al., (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al, (2002) Molec. Cell 10:895-905; Epinat et al, (2003) Nucleic Acids Res. 31 :2952-2962; Ashworth et al, (2006) Nature 441 :656-659; Paques et al, (2007) Current Gene Therapy 7:49-66; U.S. Pat. Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (-1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Pat. Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Pat. Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain embodiments, the target sites are within 600 base pairs on either side of a transcription start site (TSS) of the gene.

It is within the level of a skilled artisan to design or identify a gRNA sequence that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a sequence with minimal off-target binding to a non-target gene.

In some embodiments, the regulatory factor further comprises a functional domain, e.g., a transcriptional activator.

A In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of a heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases)) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Spl, AP-2, and CTF1 (Seipel et al, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, Cl, AP1, ARF-5, -6,-1, and -8, CPRF1, CPRF4, MYC-RP/GP, and TRAB1, See, for example, Ogawa et al, (2000) Gene 245:21-29; Okanami et al, (1996) Genes Cells 1 :87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g. type- A, nuclear localized such as MYST family members MOZ, Ybf2/Sas3, MOF, and Tip60, GNAT family members Gcn5 or pCAF, the p300 family members CBP, p300 or Rtt109 (Bemdsen and Denu (2008) Curr Opin Struct Biol 18(6):682-689). In other instances the domain is a histone deacetylase (HD AC) such as the class I (HDAC-1, 2, 3, and 8), class II (HDAC IIA (HDAC-4, 5, 7 and 9), HD AC IIB (HDAC 6 and 10)), class IV (HDAC-1 1), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39h, G9a, SETDB1, Ezh2, Set2, Dotl, PRMT1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

The process of introducing the polynucleotides described herein into cells can be achieved by any suitable technique. Suitable techniques include calcium phosphate or lipid-mediated transfection, electroporation, and transduction or infection using a viral vector. In some embodiments, the polynucleotides are introduced into a cell via viral transduction (e.g., lentiviral transduction).

Once altered, the presence of expression of any of the molecule described herein can be assayed using known techniques, such as Western blots, ELISA assays, FACS assays, and the like.

In some embodiments, the present technology provides hypoimmunogenic pluripotent cells that comprise a “suicide gene” or “suicide switch”. These are incorporated to function as a “safety switch” that can cause the death of the hypoimmunogenic pluripotent cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al, Mol. Therap. 20(10): 1932-1943 (2012), Xu et al, Cell Res. 8:73-8 (1998), both incorporated herein by reference in their entirety.)

In other embodiments, the suicide gene is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 is triggered by the administration of a chemical inducer of dimerization (CID). In some embodiments, the CID is the small molecule drug API 903. Dimerization causes the rapid induction of apoptosis. (See, WO2011146862; Stasi et al, N. Engl. J. Med 365;18 (2011); Tey et al, Biol. Blood Marrow Transplant. 13:913-924 (2007), each of which are incorporated by reference herein in their entirety.)

### K. Generation of Hypoimmunogenic Pluripotent Stem Cells

The present technology provides methods of producing hypoimmunogenic pluripotent cells. In some embodiments, the method comprises generating pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPSCs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from the group consisting of SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In general, as is known in the art, iPSCs are made from non-pluripotent cells such as, but not limited to, blood cells, fibroblasts, etc., by transiently expressing the reprogramming factors as described herein.

### L. Assays for Hypoimmunogenicity Phenotypes and Retention of Pluripotency

Once the hypoimmunogenic cells have been generated, they may be assayed for their hypoimmunogenicity and/or retention of pluripotency as is described in WO2016183041 and WO2018132783, each of which are incorporated by reference herein in their entirety.

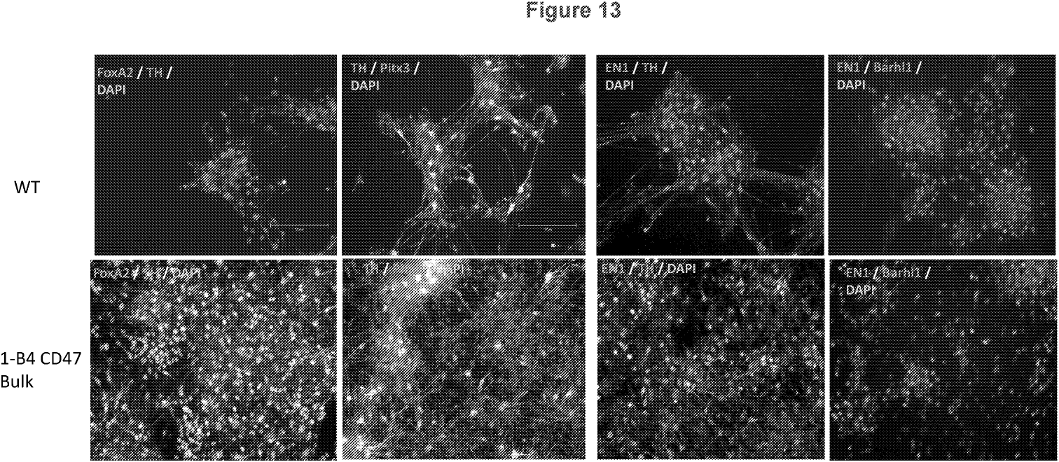

In some embodiments, hypoimmunogenicity is assayed using a number of techniques as exemplified in FIG. 13 and FIG. 15 of WO2018132783 incorporated by reference herein. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783 incorporated by reference herein.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells in accordance with the present technology and determining the expression levels of T cell activation markers in the T cells.

In vivo assays can be performed to assess the immunogenicity of the cells of the present technology. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In one embodiment, pluripotency is assayed by the expression of certain pluripotency-specific factors as generally described herein and shown in FIG. 29 of WO2018132783, which is incorporated by reference herein. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, B, C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, which is incorporated by reference herein, for example) and generally is done using either Western Blots or FACS analysis based on commercial antibodies that bind to human HLA Class II HLA-DR, DP and most DQ antigens.

In addition to the reduction of HLA I and II (or MHC I and II), the hypoimmunogenic cells have a reduced susceptibility to macrophage phagocytosis and NK cell killing. The resulting hypoimmunogenic cells “escape” the immune macrophage and innate pathways due to the expression of one or more CD47 transgenes or one or more CD24 transgenes.

In addition, any of the cells described herein inhibit microglial phagocytosis. In addition, the cells can be tested to confirm that microglial phagocytosis is inhibited. This may be assayed and assessed by using, for example, neural cell tracer dyes, Elispot, ELISA, FACS, PCR, mass cytometry (CYTOF), immunostaining, neuroinflammatory marker assays, microglial activation assays, or other assays known in the art to measure microglial phagocytosis or the functional state of microglia. Neural assessments of a recipient who has been administered the neural cells of the present technology can be performed. Non-limiting examples of useful neural assessment include structural/functional brain MRI assessment, PET imaging, PET/MRI imaging, motor function examinations, neuropsychological testing, neurocognitive assessments, cognitive and behavior evaluations, spatial learning tests, memory tests, and any clinically recognized test for evaluating a patient with any of the neurological disorders or conditions described herein. For instance, detailed descriptions of in vitro and in vivo assays and studies that are useful to evaluate microglia structure and function can be found in, e.g., Ramesha et al., J. Vis. Exp., 2005, (160), e61467; Beurner et al., Gene Therapy, 2013, 20:797-806; Pearse et al., Int J Mol Scie, 2018, 19(9), 2550; Svoboda et al., Prot Natl Acad Scie USA, 2019, 116(50):25293-25303; Mancuso et al., Nat Neurosc, 2019, 22:2111-2116; Modo et al., Brain Research, 2002, 958(1):70-82; and Xu et al., Cell Reports, 2020, 32(6):108041, as well as U.S. Pat. Nos. 9,724,432; 10,190,095; 10,279,051; 10,485,807; 10,626,369; U.S. Publ. Application No. 20200197445; and PCT Publ. Application Nos. WO2000023571; WO2003070171; WO2008089267; WO2009137674; WO2014124087; WO2018209022; WO2019246262; WO2020167822; and WO2021011936.

### M. Maintenance of Hypoimmunogenic Pluripotent Stem Cells

Once the hypoimmunogenic pluripotent stem cells have been generated, they can be maintained an undifferentiated state as is known for maintaining iPSCs. For example, the cells can be cultured on Matrigel using culture media that prevents differentiation and maintains pluripotency. In addition, they can be in culture medium under conditions to maintain pluripotency.

### N. Transplantation of Neural Cells Differentiated From Hypoimmunogenic Stem Cells

As will be appreciated by those in the art, the differentiated hypoimmunogenic pluripotent cell derivatives can be transplanted using techniques known in the art that depends on both the cell type and the ultimate use of these cells.

The neural cells can be administered in a manner that permits them to engraft to the intended tissue site and reconstitute or regenerate the functionally deficient area. For instance, neural cells can be transplanted directly into parenchymal or intrathecal sites of the central nervous system, according to the disease being treated. In some embodiments, any of the neural cells described herein including cerebral endothelial cells, neurons, dopaminergic neurons, ependymal cells, astrocytes, microglial cells, oligodendrocytes, and Schwann cells are injected into a patient by way of intravenous, intraspinal, intracerebroventricular, intrathecal, intraarterial, intramuscular, intraperitoneal, subcutaneous, intramuscular, intra-abdominal, intraocular, retrobulbar and combinations thereof. In some embodiments, the cells are injected or deposited in the form of a bolus injection or continuous infusion. In certain embodiments, the neural cells are administered by injection into the brain, apposite the brain, and combinations thereof. The injection can be made, for example, through a burr hole made in the subject’s skull. Suitable sites for administration of the neural cell to the brain include, but are not limited to, the cerebral ventricle, lateral ventricles, cisterna magna, putamen, nucleus basalis, hippocampus cortex, striatum, caudate regions of the brain and combinations thereof.

For therapeutic application, cells prepared according to the disclosed methods can typically be supplied in the form of a pharmaceutical composition comprising an isotonic excipient and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

Additional methods for administering differentiated neural cells are explained fully in the literature. See, e.g., Xi, J et al., Specification of Midbrain Dopamine Neurons from Primate Pluripotent Stem Cells. Stem Cells, 2012; 30:1655-1663; Kim et al., Biphasic Activation of WNT Signaling Facilitates the Derivation of Midbrain Dopamine Neurons from hESCs for Translational Use, Cell Stem Cell, 2021; 28:343-355; Nolbrantnet al., Generation of high-purity human ventral midbrain dopaminergic progenitors for in vitro maturation and intracerebral transplantation, Nature Protocols, 2018; 12:9 1962-1979; Xiong et al., Human Stem Cell-Derived Neurons Repair Circuits and Restore Neural Function, Cell Stem Cell, 2021, 28;1-15; and U.S. Pat. No. 10,858,625, U.S. Pat. No. 10,828,335, U.S. Pat. No. 8,597,945, U.S. Pat. Application No. 2016/0348070A1; U.S. Pat. Application No. 2019/0211306A1; WO2018035214A1; and WO2019016113A1, the disclosures of which are incorporated by reference herein in their entirely including the figures, figure descriptions and examples.

### O. Immunosuppressive Agents

In some embodiments, an immunosuppressive and/or immunomodulatory agent is not administered to the patient before the administration (e.g., the first administration) of the population of hypoimmunogenic neural cells.

In many embodiments, an immunosuppressive and/or immunomodulatory agent is administered to the patient before the first administration of the population of hypoimmunogenic neural cells. In some embodiments, an immunosuppressive and/or immunomodulatory agent is administered at least 1, 2, 3, 4, 5, 6, 7, 8, 9, 10, 11, 12, 13, 14 days or more before the first administration of the neural cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first administration of the neural cells. In some embodiments, an immunosuppressive and/or immunomodulatory agent is not administered to the patient after the first administration of the neural cells, or is administered at least 1, 2, 3, 4, 5, 6, 7, 8, 9, 10, 11, 12, 13, 14 days or more after the first administration of the neural cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first administration of the neural cells. Non-limiting examples of an immunosuppressive and/or immunomodulatory agent include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, or CD58, and antibodies binding to any of their ligands. In one embodiment, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA-4) and similar agents. In some embodiments, where an immunosuppressive and/or immunomodulatory agent is administered to the patient before or after the first administration of the neural cells, the administration is at a lower dosage than would be required for cells with MHC I and/or MHC II expression and without exogenous expression of CD47. In some embodiments, where an immunosuppressive and/or immunomodulatory agent is administered to the patient before or after the first administration of the neural cells, the administration is at a lower dosage than would be required for cells with MHC I and/or MHC II expression and without exogenous expression of CD24.

In some embodiments, an immunosuppressive and/or immunomodulatory agent is not administered to the patient before the administration of the population of hypoimmunogenic cells. In many embodiments, an immunosuppressive and/or immunomodulatory agent is administered to the patient before the first and/or second administration of the population of hypoimmunogenic neural cells. In some embodiments, an immunosuppressive and/or immunomodulatory agent is administered at least 1, 2, 3, 4, 5, 6, 7, 8, 9, 10, 11, 12, 13, 14 days or more before the administration of the neural cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first and/or second administration of the neural cells. In some embodiments, an immunosuppressive and/or immunomodulatory agent is administered at least 1, 2, 3, 4, 5, 6, 7, 8, 9, 10, 11, 12, 13, 14 days or more after the administration of the neural cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first and/or second administration of the neural cells. In some embodiments where an immunosuppressive and/or immunomodulatory agent is administered to the patient before or after the administration of the cells, the administration is at a lower dosage than would be required for cells with MHC I and/or MHC II expression and without exogenous expression of CD47. In some embodiments where an immunosuppressive and/or immunomodulatory agent is administered to the patient before or after the administration of the cells, the administration is at a lower dosage than would be required for cells with MHC I and/or MHC II expression and without exogenous expression of CD24.

### IV. EXAMPLES

### Example 1. Hypoimmunogenic Cerebral Endothelial Cell Grafts for the Treatment of Neurologic Disease

### A. Background

With the emergence of cell therapy, beneficial effects of cell-mediated vascular repair in various neurological diseases have been assumed for endothelial cells (ECs). Cerebral ECs provide structural support, control of blood flow, and regulate neurotransmitters, all parameters which are compromised in a disease state. Generally, the central nervous system (CNS) is considered immunoprivileged because of its immunologically unique niche separated from the circulation by the blood brain barrier (BBB). The complex neuroimmune interactions between transplanted cells in the brain and the local immune environment, however, have not yet been thoroughly defined. Specifically, the disruption of the BBB in the context of stereotactic cell delivery may impede its immune barrier function and make allogeneic cell grafts susceptible to rejection.

### B. Methods

### 1. Endothelial Cell (EC) Differentiation

iPSCs were plated on gelatin in 6-well plates and maintained in iPSC media. After the cells reached 60% confluency, the differentiation was started and media was changed to RPMI-1640 containing 2% B-27 minus Insulin (both Gibco) and 5 µM CHIR-99021 (Selleckchem, Munich, Germany). On day 2, the media was changed to reduced media: RPMI-1640 containing 2% B-27 minus Insulin (both Gibco) and 2 µM CHIR-99021 (Selleckchem). From day 4 to day 7, cells were exposed to RPMI-1640 EC media, RPMI-1640 containing 2% B-27 minus Insulin plus 50 ng/ml mouse vascular endothelial growth factor (mVEGF; R&D Systems, Minneapolis, MN), 10 ng/ml mouse fibroblast growth factor basic (mFGFb; R&D Systems), 10 µM Y-27632 (Sigma-Aldrich, Saint Louis, MO), and 1 µM SB 431542 (Sigma-Aldrich). Endothelial cell clusters were visible from day 7 and cells were maintained in Endothelial Cell Basal Medium 2 (PromoCell, Heidelberg, Germany) plus supplements, 10% FCS hi (Gibco), 1% Pen/Strep, 25 ng/ml VEGF, 2 ng/ml FGFb, 10 µM Y-27632 (Sigma-Aldrich), and 1 µM SB 431542 (Sigma-Aldrich). The differentiation process was completed after 21 days and undifferentiated cells detached during the differentiation process. For purification, cells went through MACS purification according the manufactures’ protocol using anti-CD15 mAb-coated magnetic microbeads (Miltenyi, Auburn, CA) for negative selection. The highly purified ECs in the flow-through were cultured in EC media as described above. TrypLE was used for passaging the cells 1:3 every 3 to 4 days.

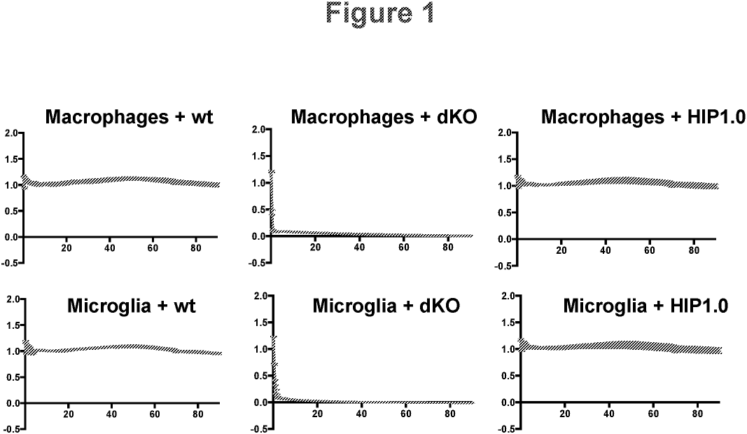

### 2. Microglia Killing Phagocytosis Assay by XCelligence (FIG. 1)

Microglia killing assay was performed on the XCelligence platform (ACEA BioSciences, San Diego, CA.). 96-well E-plates (ACEA BioSciences) were coated with collagen (Sigma-Aldrich) and 4 × 10⁵ wt, B2m^{-/-}Ciita^{-/-}, or B2m^{-/-}Ciita^{-/-} CD47 tg ECs and were plated in 100µl cell specific media. After the Cell Index value reached 0.7, microglia was added with an E:T ratio of 1:1. As a negative control, cells were treated with 2% TritonX-100 (data not shown). Data were standardized and analyzed with the RTCA software (ACEA).

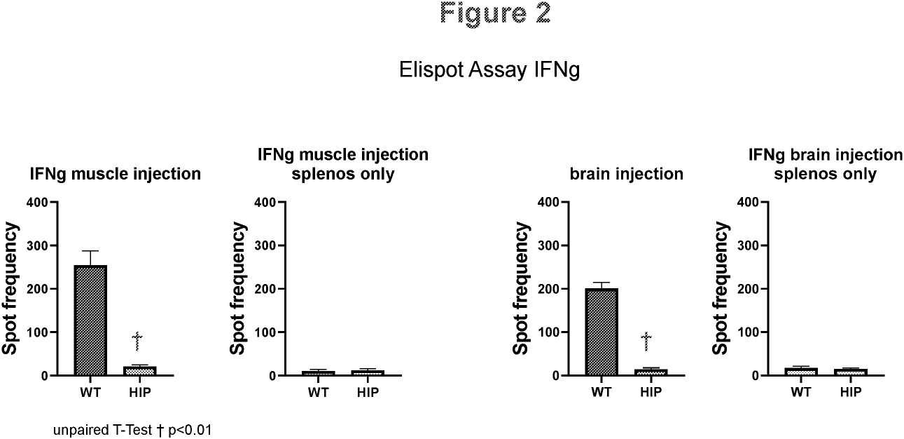

### 3. T Cell Elispot (FIG. 2)

1×10⁶ wt or B2m^{-/-}Ciita^{-/-} CD47 tg ECs were injected into the striatum of the brain or limb muscle and spleens were recovered after 5 days for T cell isolation. For T-cell specific Elispot Assay, T cells from BALB/c mice were co-cultured with wt or B2m^{-/-}Ciita^{-/-} CD47 tg ECs and their IFN-y release was measured. Mitomycin-treated (50 µg/ml for 30 min) stimulator cells were incubated with T cells for 24 h and IFN-y spot frequencies were enumerated using an Elispot plate reader. T cells without stimulator cells were used as background controls.

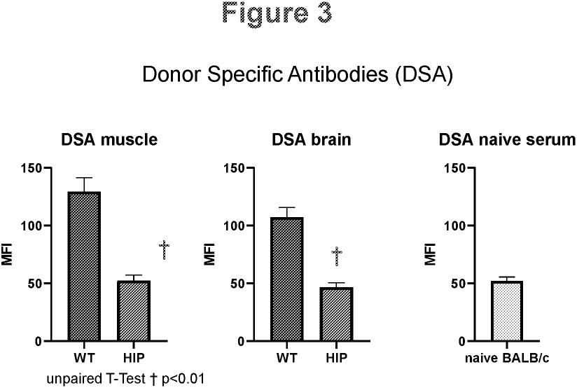

### 4. Donor-Specific Antibodies (FIG. 3)

Donor-specific antibodies (DSAs) were detected by FACS analysis. The serum of Balb/c 5 days after transplantation of wt or B2m^{-/-}Ciita^{-/-} CD47 tg ECs was incubated with graft cells and the binding of graft-specific antibodies was quantified. Only IgM antibodies were analysed because of their known rapid surge within 5 days upon allogeneic stimulation. IgM antibodies were stained by incubation of the cells with a PE-conjugated goat antibody specific for the Fc portion of mouse IgM (BD Biosciences). Cells were washed and then analyzed on an Attune system. Fluorescence data were expressed as MFI.

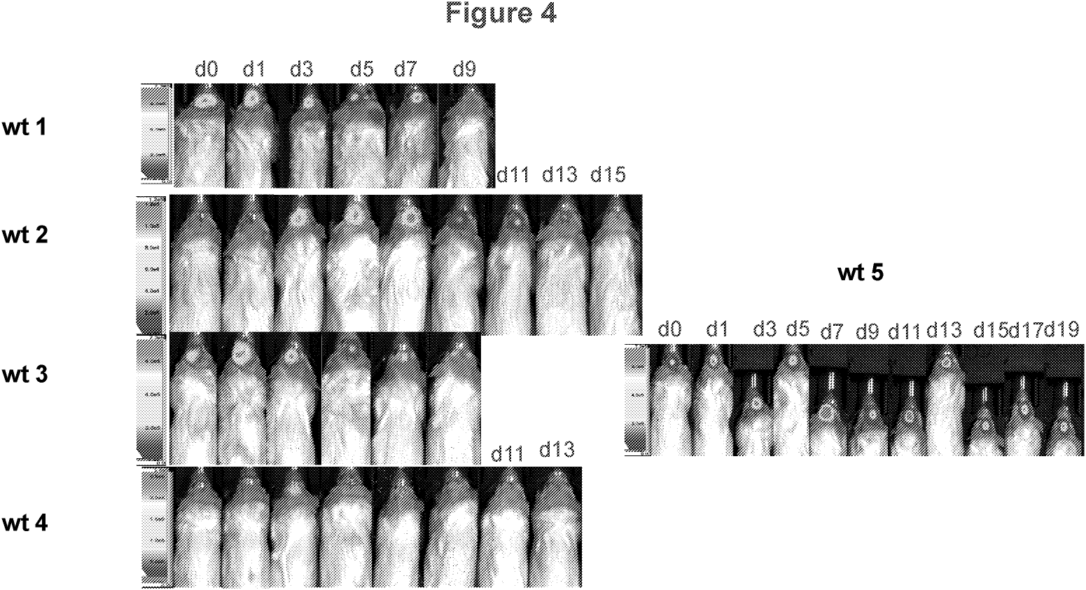

### 5. Bioluminescence Imaging (BLI; FIGS. 4-7)

For BLI, D-luciferin firefly potassium salt (375 mg/kg) (Biosynth AG) dissolved in sterile PBS (pH 7.4) (Gibco, Invitrogen) was injected i.p. (250 µl per mouse) into anesthetized mice. Animals were imaged using the ami HT (Spectral Instruments Imaging, Tucson, AZ) Region of interest (ROI) bioluminescence was quantified in units of maximum photons per second per centimeter square per steradian (p/s/cm2/sr). The maximum signal from an ROI was measured using Living Image software (Media Cybernetics). Mice were monitored on day 0, day 1, and every other day until cells were rejected or up to 28 days.

### C. Results

To circumvent immune rejection after allogeneic transplantation, hypoimmunogenic iPSCs were engineered to evade allogeneic immune recognition by inactivating the B2M and CIITA genes to deplete both major histocompatibility complex (MHC) class I and II epitopes using CRISPR-Cas9 and guides and achieved CD47 overexpression to protect from innate immune killing.

Microglia act as the first line of active immune defense in the brain. Using an in vitro setup, it was demonstrated that hypoimmunogenic ECs (overexpressing CD47) are directly protected from microglia phagocytosis (FIG. 1). In the experiment, the effect of CD47 expression and microglia inhibition was evaluated. Wildtype human ECs, double knockout (B2M-/-CIITA-/-) ECs, and dKO/CD47Tg (B2M-/-CIITA-/- CD47 tg) ECs were co-cultured with allogeneic human macrophages or microglia. The data show that CD47 protected double knockout (dKO) cells from macrophage killing (FIG. 1, top panel). CD47 also protected double knockout (dKO) cells from microglia killing (FIG. 1, bottom panel).

The immune fate of mouse C57BL/6 iPSC-derived ECs in the brain after injection into the striatum was investigated. When injected into allogeneic BALB/c mice, wild type (wt) ECs triggered a vigorous lymphocytic interferon-gamma immune response in the spleen as well as production of donor-specific antibodies (DSA) in the serum (FIGS. 2 and 3). After injection into the striatum of allogeneic, fully MHC-mismatched BALB/c mice, no systemic cellular or antibody response was observed (FIGS. 2 and 3).

FIG. 2 shows a comparison of immune response after allogeneic miPSC-derived endothelial transplantation into the brain versus muscle. Injections of wild-type ECs into the brain induced a T cell response, as assays by IFN-gamma Elispot assay. dKO/CD47Tg cells did not induce a systemic T cell response, which is also seen in other organs including muscle.

FIG. 3 shows a comparison of immune response after allogeneic miPSC-derived endothelial transplantation into the brain versus muscle, as determined by donor specific antibodies (DSA). Injections of the wild-type ECs into the brain induced a donor-specific antibody response. In contrast, dKO/CD47Tg cells did not induce DSA production, which is also seen in other organs including muscle.

Wild-type miPSC-derived endothelial cells were transplanted into the striatum of healthy allogeneic BALB/c mice brains. Bioluminescence imaging showed that wild-type ECs were rejected within 15 days (4 out of 5 mice), as depicted in FIG. 4.

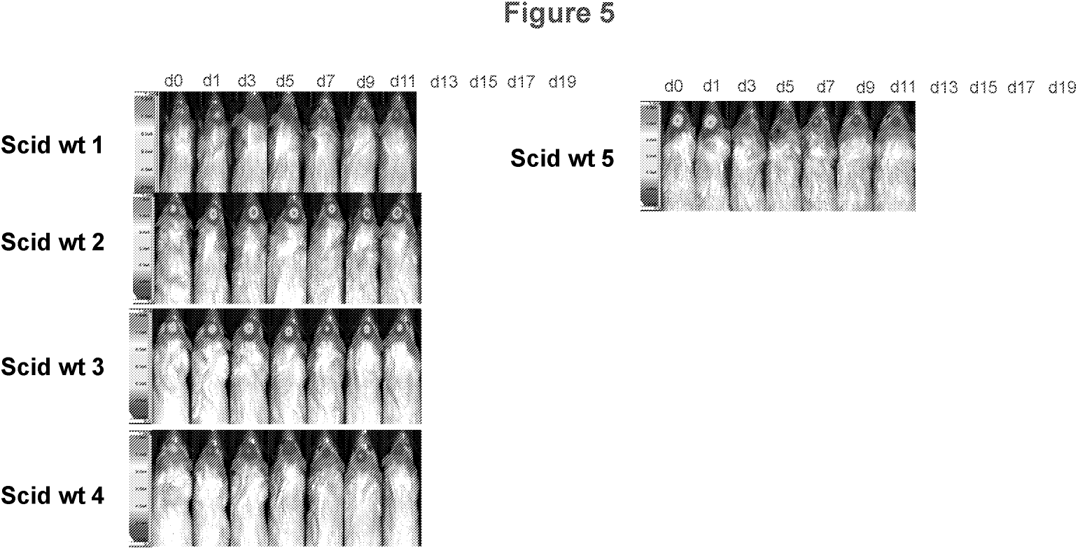

Wild-type ECs, however, survived in healthy immunocompromised SCID-beige mice (FIG. 5). WT cells survived for at least 11 days in immunocompromised mice.

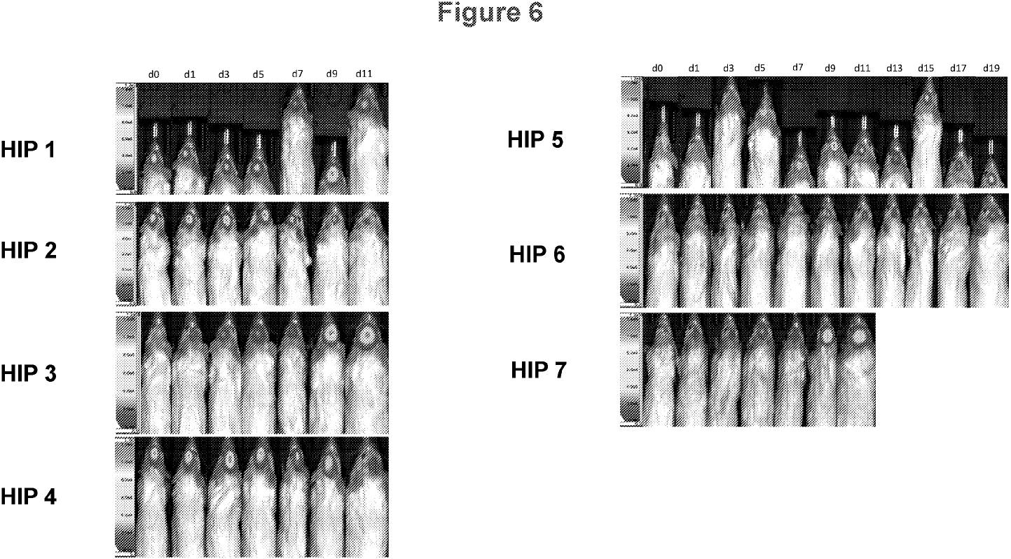

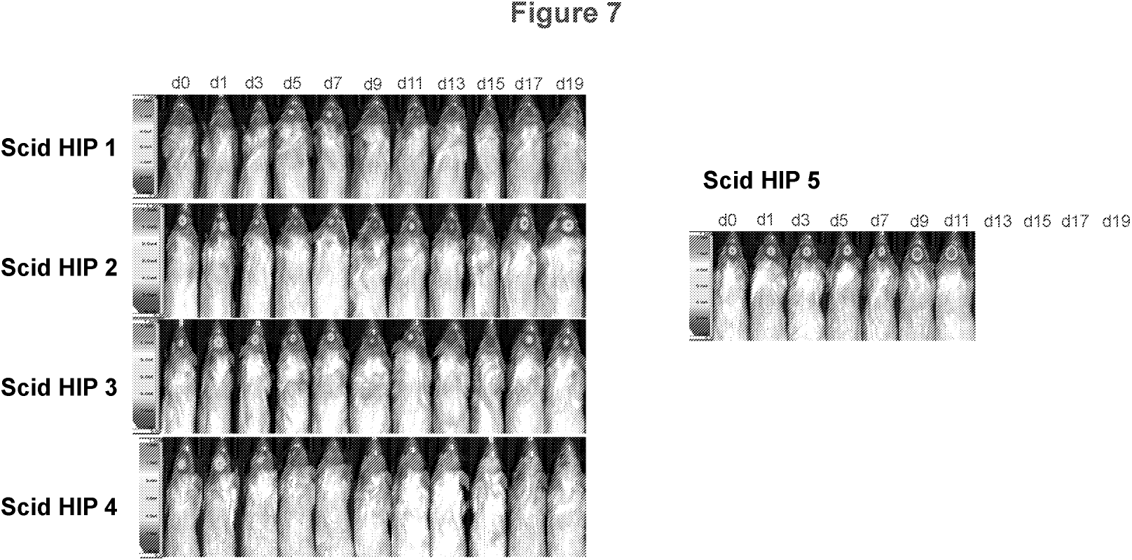

Hypoimmune EC grafts steadily survived the 19 day follow-up in allogeneic mice (7 out of 7) without using any immunosuppression (FIG. 6). dKO/CD47Tg ECs were transplanted into the striatum of healthy allogeneic BALB/c mice brains. Such cells also survived in healthy immunocompromised SCID-beige mice (FIG. 7). dKO/CD47Tg ECs survived for at least 19 days in the immunocompromised mice.

The data demonstrates that CD47 overexpression is protective against microglial phagocytosis. Thus, hypoimmune cells expressing CD47 are suitable in therapeutic CNS strategies. The results also show that the brain is not an “immunoprivileged” organ for stem cell transplantation approaches.

### D. Conclusion

In summary. hypoimmunogenic cell compositions described herein were shown to evade immune cell activation in various organ system, including the brain.

### Example 2. Hypoimmunogenic Cerebral Endothelial Cell Grafts in a Stroke Model

Stroke is one of the leading causes of death and disability worldwide. Novel therapies are needed to ameliorate ischemic stroke. This example describes a study to investigate the use of in vitro differentiated endothelial cells from pluripotent stem cells to treat stroke.

### A. Methods

### 1. Endothelial Cell (EC) in Vitro Differentiation

iPSCs are plated on gelatin in 6-well plates and maintained in iPSC media. After the cells reached 60% confluency, the differentiation is started and media is changed to RPMI-1640 containing 2% B-27 minus Insulin (both Gibco) and 5 µM CHIR-99021 (Selleckchem, Munich, Germany). On day 2, the media is changed to reduced media: RPMI-1640 containing 2% B-27 minus Insulin (both Gibco) and 2 µM CHIR-99021 (Selleckchem). From day 4 to day 7, cells are exposed to RPMI-1640 EC media, RPMI-1640 containing 2% B-27 minus Insulin plus 50 ng/ml mouse vascular endothelial growth factor (mVEGF; R&D Systems, Minneapolis, MN), 10 ng/ml mouse fibroblast growth factor basic (mFGFb; R&D Systems), 10 µM Y-27632 (Sigma-Aldrich, Saint Louis, MO), and 1 µM SB 431542 (Sigma-Aldrich). Endothelial cell clusters are visible from day 7 and cells are maintained in Endothelial Cell Basal Medium 2 (PromoCell, Heidelberg, Germany) plus supplements, 10% FCS hi (Gibco), 1% Pen/Strep, 25 ng/ml VEGF, 2 ng/ml FGFb, 10 µM Y-27632 (Sigma-Aldrich), and 1 µM SB 431542 (Sigma-Aldrich). The differentiation process is completed after 21 days and undifferentiated cells detach during the differentiation process. For purification, cells undergo MACS purification according the manufacturer’s protocol using anti-CD15 mAb-coated magnetic microbeads (Miltenyi, Auburn, CA) for negative selection. The highly purified ECs in the flow-through are cultured in EC media as described above. TrypLE is used for passaging the cells 1:3 every 3 to 4 days.

### 2. Microglia Killing Phagocytosis Assay by XCelligence

Microglia killing assay is performed on the XCelligence platform (ACEA BioSciences, San Diego, CA.). 96-well E-plates (ACEA BioSciences) are coated with collagen (Sigma-Aldrich) and 4 × 10⁵ wt, B2m^{-/-}Ciita^{-/-}, B2m^{-/-}Ciita^{-/-} CD47 tg, or B2m^{-/-}Ciita^{-/-} CD24 tg ECs and are plated in 100µl cell specific media. After the Cell Index value is reached 0.7, microglia is added with an E:T ratio of 1:1. As a negative control, cells are treated with 2% TritonX-100 (data not shown). Data are standardized and analyzed with the RTCA software (ACEA).

### 3. T Cell Elispot

1x10⁶ wt, B2m^{-/-}Ciita^{-/-}, B2m^{-/-}Ciita^{-/-} CD47 tg, or B2m^{-/-}Ciita^{-/-} CD24 tg ECs are injected into the striatum of the brain or limb muscle and spleens are recovered after 5 days for T cell isolation. For T-cell specific Elispot Assay, T cells from BALB/c mice are co-cultured with wt, B2m^{-/-}Ciita^{-/-}, B2m^{-/-}Ciita^{-/-} CD47 tg, or B2m^{-/-}Ciita^{-/-} CD24 tg ECs and their IFN-γ release is measured. Mitomycin-treated (50 µg/ml for 30 min) stimulator cells are incubated with T cells for 24 h and IFN-y spot frequencies are enumerated using an Elispot plate reader. T cells without stimulator cells are used as background controls.

### 4. Donor-Specific Antibodies

Donor-specific antibodies (DSAs) are detected by FACS analysis. The serum of Balb/c 5 days after transplantation of wt, B2m^{-/-}Ciita^{-/-}, B2m^{-/-}Ciita^{-/-} CD47 or B2m^{-/-} Ciita^{-/-} CD24 tg ECs are incubated with graft cells and the binding of graft-specific antibodies is quantified. Only IgM antibodies are analysed because of their known rapid surge within 5 days upon allogeneic stimulation. IgM antibodies are stained by incubation of the cells with a PE-conjugated goat antibody specific for the Fc portion of mouse IgM (BD Biosciences). Cells are washed and then analyzed on a Attune system. Fluorescence data were expressed as MFI.

### 5. Bioluminescence Imaging (BLI)

For BLI, D-luciferin firefly potassium salt (375 mg/kg) (Biosynth AG) dissolved in sterile PBS (pH 7.4) (Gibco, Invitrogen) is injected i.p. (250 µl per mouse) into anesthetized mice. Animals are imaged using the ami HT (Spectral Instruments Imaging, Tucson, AZ) Region of interest (ROI) bioluminescence is quantified in units of maximum photons per second per centimeter square per steradian (p/s/cm2/sr). The maximum signal from an ROI is measured using Living Image software (Media Cybernetics). Mice are monitored on day 0, day 1, and every other day until cells are rejected or up to 28 days.

### 6. Mouse Model of Ischemic Stroke

In the middle cerebral artery occlusion (MCAO) mouse model, mice are anesthetized using a mixture of 70% NO₂, 30% O₂, and 2.0-2.5% isoflurane using an animal general anesthesia machine. The left common carotid artery is exposed using a midline skin incision and the origin of the middle cerebral artery is occluded using an 8-0 nylon monofilament coated with Provil novo (Heraeus, Hanau, Germany). After 2 hours of MCAO, the filament is removed to establish reperfusion under brief anesthesia. After the procedure, the mice are kept in the same conditions as the preoperative environment (24 ± 2° C.; 12-hour light/dark cycle).

### 7. Assessment of Cerebral Infarction

The cerebral infarction is assessed by quantifying the infarct volume. Forebrains of the mice are removed under deep anesthesia with isoflurane 24 hours after the induction of MCAO. Coronal sections are prepared and stained using a 2% TTC solution for 30 minutes. The stained sections are digitally imaged and analyzed using image-processing software.

### 8. Behavior Assessment

Behavioral assessments are made of the mice after the induction of MCAO. For example, 6 behaviors can be evaluated including: spontaneous activity for 5 minutes, symmetry of the four limbs during movement, symmetry of forelimb outstretching, climbing ability, body proprioception, and response to vibrissae touch. Each behavior is scored on a point scale and a total score is calculated. Lower scores indicate more severe neurological deficits.

### 9. Assessment of Cerebral Blood Flow

Cerebral blood flow (CBF) can be measured in the cerebral ischemic mice using laser Doppler flowmetry.

Mice are placed in the prone position under brief anesthesia with 2.0-3.0% isoflurane, and the skull is exposed using a linear skin incision. The skull is exposed to a 780-nm semiconductor laser. The reflected light, which is linearly polarized, is detected using a charge-coupled device camera placed above the head through a hybrid filter. The hybrid filter is used to preclude the detection reflected light from the surface of the tissue and to enable stable and specific measurements. The raw speckle images are videotaped to evaluate the speckle contrast, which indicates the velocity and numbers of moving red blood cells. The average of 20 consecutive raw speckle images is used to obtain one blood flow image. The red color indicates the higher cerebral blood flow. The images are analyzed using the software installed in a laser speckle imaging system. Oval regions of interest (ROIs) are created at the ipsilateral and contralateral middle cerebral artery (MCA) areas. Relative blood flow in the MCA area is calculated by averaging the blood flow of the ROIs, and the ratio of ipsilateral CBF to contralateral CBF is calculated. CBF is measured just before MCAO, just after MCAO, before reperfusion, after reperfusion, before injection of the test neural cells, and after injection of the test neural cells.

Additional details related to mouse models of ischemic stroke and methods of evaluating such mice can be found in, for example, Yamauchi et al., Scientific Reports, 2017, 7:12088, the disclosure is herein incorporated by reference in its entirety.

### B. Experimental Plan

The endothelial cells (ECs) generated according to the methods outlined above are evaluated in a mouse model of stroke - a focal cerebral ischemia mouse model. Cerebral endothelial cells are administered to mice with cerebral ischemia. In some cases, the cells are injected via intraparenchymal, intracerebroventricular, or intrathecal (e.g., cisternal or lumbar) administration.

In this study iPSCs are differentiated to generate wt, B2m^{-/-}Ciita^{-/-}, B2m^{-/-}Ciita^{-/-} CD47 tg, or B2m^{-/-}Ciita^{-/-} CD24 tg ECs according to the method described above. The cells are injected into a focal cerebral ischemia mouse model.

Briefly, mice are anesthetized in a knockdown chamber using 3% isoflurane + oxygen. When the animal has lost consciousness, its weight is recorded and the top of head of the animal is shaved from level of eyes to behind the ears. The mouse is transferred to a stereotaxic device. The amount of anesthesia is reduced to 1.5% isoflurane. The shaved area is disinfected with 3% chlorhexidine gluconate.

A midline incision is made approximately 1 cm midway between the eyes/ears. Subcutaneous connective tissue is separated from the cranium. 70% EtOH is applied to the cranium to clean. Using a microdrill a small hole is drilled at coordinates: AP= -1.2 mm and ML= -1.1 mm from Bregma, being careful not to drill through the dura mater.

A Hamilton syringe fit with a 23G needle is loaded with ECs. The microdrill is replaced with UltraMicroPump 3T (UMP3T; World Precision Instruments) and the Hamilton syringe is loaded into the UMP3T. Infusion parameters are set to about 500 nl/min. About 6.5 µl (total 1 million cells) are infused into MFB (medial forebrain) through the previously drilled hole at a depth of DV=-5.00 mm. After infusion, the needle is slowly retracted.

The skin incision is closed using a 7-0 absorbable suture and an inverted simple interrupted stitch. The animal is removed from the stereotaxic device and placed in warm recovery cage. Once animal has regained consciousness and is sternal, it is returned to its home cage. The animal’s weight is monitored for a minimum of each day for three days.

Effects of the transplanted cerebral ECs on the focal cerebral ischemia mice are evaluated according to methods recognized by those skilled in the art. For example, changes or differences in behavior, cerebral infarct, cerebral blood flow, and the like can be evaluated in focal cerebral ischemia mice administered cerebral ECs - wt, B2m^{-/-}Ciita^{-/-}, or B2m^{-/-}Ciita^{-/-} CD47 tg ECs.

### Example 3. Hypoimmunogenic Dopaminergic Neuron Grafts in a Parkinson’s Disease Model

This example describes a study to investigate the use of in vitro differentiated dopaminergic (DA) neurons from pluripotent stem cells to treat Parkinson’s disease.

### A. Methods

### 1. Dopaminergic Neurons (including DA Neuron Progenitors) in Vitro Differentiation

iPSCs (e.g., wt, B2m^{-/-}Ciita^{-/-}, B2m^{-/-}Ciita^{-/-} CD47 tg, or B2m^{-/-}Ciita^{-/-} CD24 tg iPSCs) are plated onto LM511-E8 coated 6-well plates and maintained in iPSC media. After the cells are confluent, the maintenance medium is changed to a differentiation medium comprising GMEM supplemented with 8% KSR, 0.1 mM MEM non-essential amino acids, 1 mM sodium pyruvate, and 0.1 mM 2-mercaptoethanol, and additives including 10 nM LDN193189 and 500 nM A83091. From day 1 to day 7 of differentiation, 2 µM purmorphamine and 100 ng/ml FGF8 are also added to the differentiation medium. From day 2 to day 12 of differentiation, the cells are culture in differentiation medium supplemented with 3 µM CHIR99021.

The cells are sorted on day 12 using standard procedures to select for and isolated CORIN (a floor plate marker) positive cells. The sorted cells are plated on low cell adhesion plates and cultured in neural differentiation medium comprising neurobasal medium supplement with B27 supplement, 2 mM L-glutamine, 10 ng/ml GDNF, 200 mM ascorbic acid, 20 ng/ml BDNF, and 400 µM dbcAMP. For the initial plating after sorting, the neural differentiation medium also includes 30 µM Y-27632.

### 2. Analysis of DA Neuron Progenitors Derived From Pluripotent Stem Cells

Differentiated DA neurons and progenitors thereof are assessed by immunostaining for DA neuronal markers including FOXA2, NURR1, TUJ1, and other neural markers including PAX6 and SOX1. Electrophysiological analysis is performed using standard whole-cell patch-claim recordings known by those skilled in the art (see, e.g., Kikuchi et al., Nature, 2017, 548, 592-596).

### 3. PD Model in Monkeys

Monkeys are injected intravenously with MPTP hydrochloride twice a week until they present with Parkinsonian symptoms such as, but not limited to, tremor, bradykinesia, and impaired balance. Monkeys are transplanted with DA neuron progenitors differentiated from iPSCs as outlined above. The DA neuron progenitors are stereotactically transplanted into the putamen of the MPTP-treated monkeys bilaterally.

The animals are observed and evaluated periodically using techniques and methods for evaluating PD symptoms and functions of the transplanted cells. In some instances, the methods include MRI scanning and analysis, PET analysis, video analysis and behavioral analysis, L-DOPA tests, and the like (see, e.g., Kikuchi et al., Nature, 2017, 548, 592-596).

### B. Experimental Plan

The DA neuron progenitors generated from pluripotent stem cells are evaluated in a monkey model of Parkinson’s Disease (PD). In this study, iPSCs are differentiated to generate wt, B2m^{-/-}Ciita^{-/-}, B2m^{-/-}Ciita^{-/-} CD47 tg, or B2m^{-/-}Ciita^{-/-} CD24 tg DA neuron progenitors according to the method described above. The cells are injected into the brain of the monkey PD model.

Effects of the transplanted DA neurons are evaluated according to methods recognized by those skilled in the art. For example, changes or differences in behavior, movement, PD symptoms, dopaminergic function, and the like can be evaluated in monkeys administered the DA neurons - wt, B2m^{-/-}Ciita^{-/-}, B2m^{-/-}Ciita^{-/-} CD47 tg, or B2m^{-/-}Ciita^{-/-} CD24 tg DA neurons.

### Example 4. Pilot Study for Surgical Targeting of Cholera Toxin Subunit B (CTB) Tracer into the CNS of Mouse Models

This example describes a study to verify accuracy of surgical targeting of a fluorophore-labeled cholera toxin subunit B (CTB) tracer into mouse brains. The initial experiments are performed in C57BL/6 mice and the follow-on experiments are performed in NSG® mice.

Method - Striatal injection of a CTB tracer labelled with a fluorophore for visualization of the mouse brain by microscopy

The CTB tracer is used to visualize neurons at the injection site in the recipient mouse brain by using a microscope or scanner. In the first experiment, the CTB tracer is injected into the striatum of C57BL/6 mice (5 mice per group). The retrograde labelled neural cells are visualized at the injection site 5 days after injection. In the second experiment, the CTB tracer is injected into the striatum of NSG® mice. There are 5 mice per group and the retrograde labelled neural cells are visualized 5 days after injection.

### Example 5. Pilot Study- Surgical Targeting

This example describes a study to demonstrate engraftment of non-human primate (NHP) dopamine neurons or progenitors thereof in the brain of a recipient mouse. The dopamine (DA) neurons and progenitors are produced by differentiation of NHP induced pluripotent stem cells (iPSCs).

Methods - Bilateral injections of NHP iPSC-derived DA neuronal progenitors or neuroblast cells into the striatum of NSG® mice

Dopamine neuronal progenitors and neuroblast cells are differentiated from NHP iPSCs by any method recognized by those in the art. The dopamine neuronal progenitors and neuroblast cells are characterized prior to injection into the striatum of the recipient NSG® mice. About 150,000 dopamine neuronal progenitors and neuroblast cells are bilaterally injected into the mice.

At day 0 and day 7, striatal dissection is performed and brain samples are processed for qPCR of primate Alu content to detect the injected cells. Overall cell survival of the injected cells is determined at both timepoints.

At four months after injection (n=10 injection sites), the injected brains of the mice are sectioned and the samples undergo immunohistochemistry to detect the engrafted neurons. The presence of tyrosine hydroxylase positive (TH+) neurons and FoxA2 DA neurons, is investigated. The engrafted neurons are also detected by H&E staining and immunostaining of the mouse brain samples with an antibody that specifically detects primate or human nuclei.

### Example 6. Hypoimmunogenic iPSC Differentiation and Neuronal Maturation

This example describes a study to examine iPSC pluripotency and neuronal maturation of neuron differentiated from the hypoimmunogenic iPSCs. Expression of pluripotency genes were assessed in wild-type iPSCs, B2M-/-/CIITA-/- (dKO) iPSCs and B2M-/-/CIITA-/-CD47Tg (dKO/CD47Tg) iPSCs.

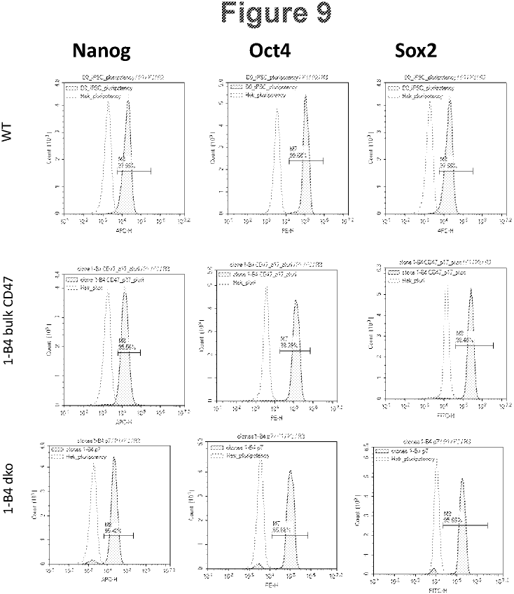

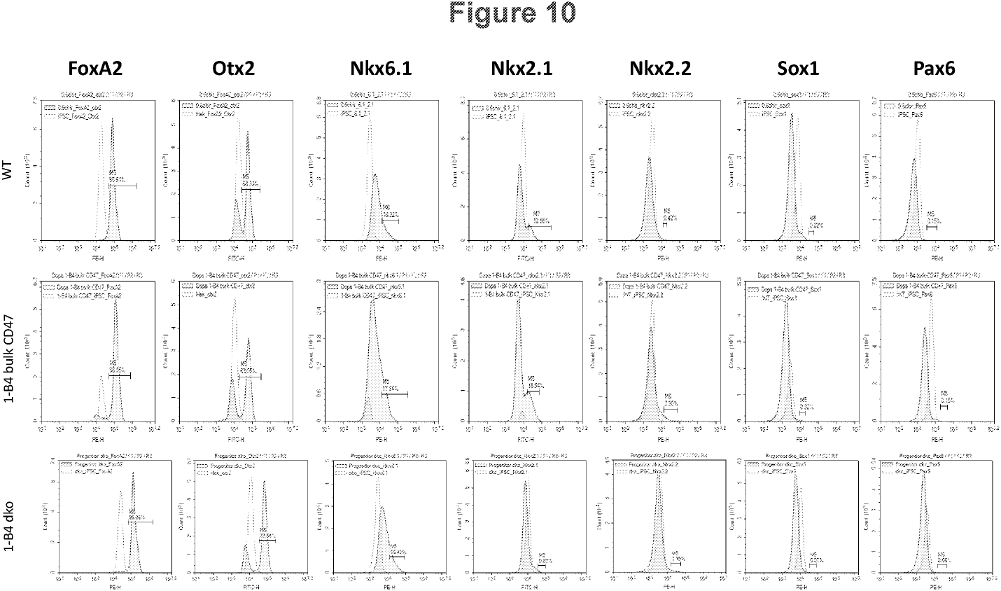

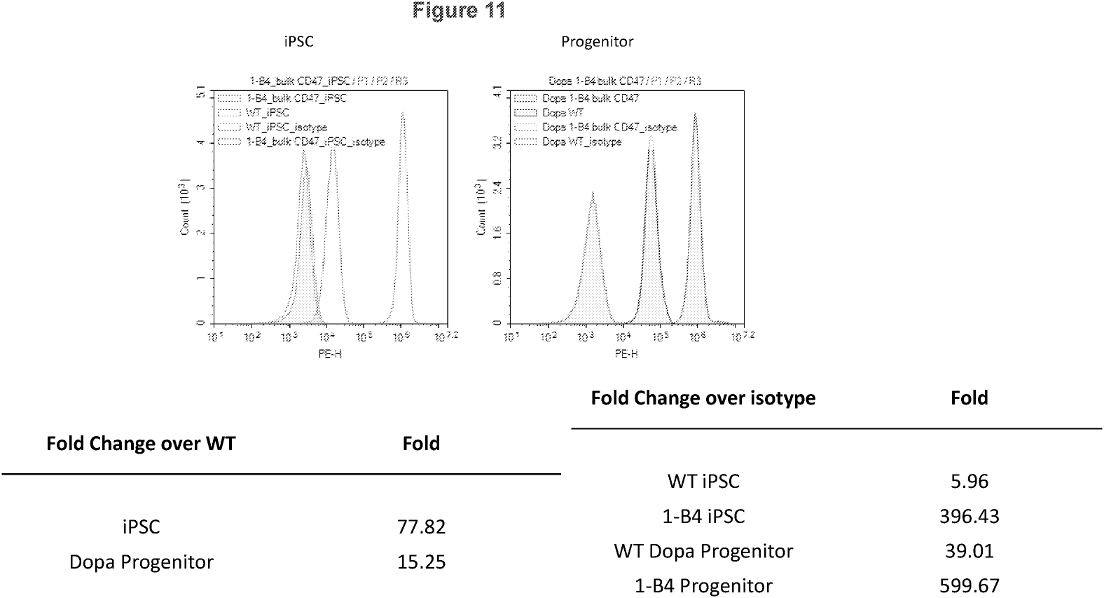

Flow cytometry was used to examine expression of Nanog, Oct4, and Sox2 in clone 1-B4 double knock-out, clone 1-B4 CD47 bulk, and wild-type cells (FIG. 9). Flow cytometry was also used to measure FoxA2, Otx2, Nkx6.1, Nkx2.2, Sox1, and Pax6 expression in dopamine progenitor cells differentiated from the clone 1-B4 double knock-out iPSCs, the clone 1-B4 CD47 bulk iPSCs, and the wild-type iPSCs (FIG. 10). Flow cytometry was also used to compare CD47 stability between wild-type iPSCs and dopamine progenitor cells differentiated therefrom, as well as clone 1-B4 CD47 bulk iPSCs and dopamine progenitor cells differentiated therefrom (FIG. 11).

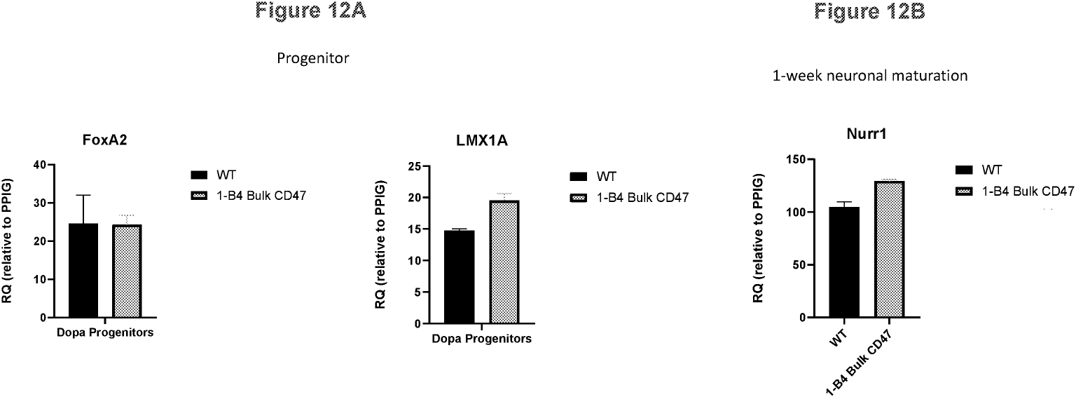

Gene expression analysis was performed on wild-type dopamine progenitor cells differentiated from wild-type iPSCs and dopamine progenitor cells differentiated from clone 1-B4 bulk CD47 cells. Expression of dopamine progenitor markers such as FoxA2 and LMX1A (FIG. 12A) and neuronal maturation markers such as Nurr1 was determined (FIG. 12B). Neuronal maturation markers were detected in neural differentiated cells at week 1 of the neuronal maturation process.

Immunofluorescence (IF) was performed on neuronal cells at week 2 of the neuronal maturation process. FoxA2, tyrosine hydroxylase (TH), engrailed-1 (EN1), pituitary homeobox 2 (Pitx2) and barH-like 1 (Barh1) staining was analyzed in the neuronal cells differentiated from either wild-type iPSCs or 1-B4 CD47 Bulk wild-type iPSCs (FIG. 13). The maturing dopamine neurons were TH+ cells. And, the maturing neuronal cells were not positive for EN1, Pitx2, and Barh1.

All headings and section designations are used for clarity and reference purposes only and are not to be considered limiting in any way. For example, those of skill in the art will appreciate the usefulness of combining various aspects from different headings and sections as appropriate according to the spirit and scope of the present technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A method for inhibiting microglial phagocytosis of a population of neural cells administered in a patient comprising administering to the patient a therapeutically effective amount of a population of neural cells comprising an exogenous CD47 polypeptide and reduced expression of MHC class I and/or MHC class II human leukocyte antigens.

2. The method of claim 1, wherein the population of neural cells comprises reduced expression of MHC class I or MHC class II human leukocyte antigens.

3. The method of claim 1, wherein the population of neural cells comprises reduced expression of MHC class I and MHC class II human leukocyte antigens.

4. The method of any one of claims 1-3, wherein the administering comprises grafting the population of neural cells into the patient’s central or peripheral nervous system.

5. The method of claim 4, wherein the grafting comprises injecting the population of neural cells into the patient.

6. The method of claim 4 or 5, wherein the grafting comprises disrupting the patient’s blood-brain barrier.

7. The method of any one of claims 1-6, wherein the population of neural cells exhibits long-term survival after the disruption of the patient’s blood-brain barrier.

8. The method of any one of claims 1-7, wherein the population of neural cells exhibits long-term function after the disruption of the patient’s blood-brain barrier.

9. The method of any one of claims 1-8, wherein the population of neural cells maintains long-term survival in the patient after the patient experiences a subsequent disruption of the patient’s blood-brain barrier that is secondary to a neurological disorder or condition.

10. The method of any one of claims 1-9, wherein the population of neural cells maintains long-term function in the patient after the patient experiences a subsequent disruption of the patient’s blood-brain barrier that is secondary to a neurological disorder or condition.

11. The method of any one of claims 1-10, wherein the subsequent disruption of the patient’s blood-brain barrier is due to an infection or a stroke.

12. The method of any one of claims 1-11, wherein the population of neural cells survives and/or functions in the patient for at least one month, two months, three months, four months or more after administration.

13. The method of any one of claims 1-12, wherein the patient is not administered an immunosuppressive agent before administration of the population of neural cells.

14. The method of any one of claims 1-13, wherein the patient is not administered an immunosuppressive agent after administration of the population of neural cells.

15. The method of any one of claims 1-14, wherein the patient requires a reduced level of immunosuppression or is substantially free of immunosuppression.

16. The method of any one of claims 1-15, wherein the neural cell is selected from the group consisting of a cerebral endothelial cell, a neuron, an ependymal cell, an astrocyte, a microglial cell, an oligodendrocyte, a Schwann cell, a progenitor thereof, and a precursor thereof.

17. The method of any one of claims 1-15, wherein the neural cell is a neural progenitor cell.

18. The method of any one of claims 1-15, wherein the neural cell is a glial progenitor cell.

19. The method of any one of claims 1-15, wherein the neural cell is a neuronal progenitor cell.

20. The method of any one of claims 1-19, wherein the microglial phagocytosis is associated with a neurological disorder or condition.

21. The method of any one of claims 9-20, wherein the neurological disorder or condition is selected from the group consisting of stroke, amyotrophic lateral sclerosis (ALS), cerebral hemorrhage, Parkinson’s disease, epilepsy, spinal cord injury, childhood hereditary leukodystrophies, congenital dysmyelination, Pelizaeus-Merzbacher disease, metabolic leukodystrophies, vanishing white matter disease, adrenoleukodystrophy, Canavan’s Disease, lysosomal storage diseases, Tay-Sachs disease, Sandhoff’s disease, Krabbe’s disease, Batten’s disease, metachromatic leukodystrophy, cerebral palsy, periventricular leukomalacia, spastic diplegias of prematurity, age-related white matter loss, subcortical dementia, vascular Leukoencephalopathies, subcortical stroke, diabetic leukoencephalopathy, hypertensive leukoencephalopathy, spinal cord injury, autoimmune demyelination, progressive multiple sclerosis, transverse myelitis, inflammatory demyelination, radiation toxicity, neurodegenerative diseases, Huntington’s Disease, frontotemporal dementia, and cerebrovascular disorders.

22. The method of any one of claims 9-20, wherein the neurological disorder or condition is Pelizaeus-Merzbacher disease.

23. The method of any one of claims 9-20, wherein the neurological disorder or condition is progressive multiple sclerosis.

24. The method of any one of claims 9-20, wherein the neurological disorder or condition is Huntington’s Disease.

25. The method of any of claims 1-24, wherein the population of neural cells express CD47 at a higher level than in an unmodified pluripotent cell or in a unmodified neural cell.

26. The method of claims 1-25, wherein the population of neural cells express a suicide gene that is activated by a trigger that causes the neural cell to die.

27. A method for inhibiting microglial phagocytosis of a population of neural cells administered in a patient comprising administering to the patient a therapeutically effective amount of a population of neural cells comprising an exogenous CD47 polypeptide and reduced expression of B2M and/or CIITA.

28. The method of claim 27, wherein the population of neural cells comprises reduced expression of B2M or CIITA.

29. The method of claim 27, wherein the population of neural cells comprises reduced expression of B2M and CIITA.

30. The method of any one of claims 27-29, wherein the administering comprises grafting the population of neural cells into the patient’s central or peripheral nervous system.

31. The method of claim 30, wherein the grafting comprises injecting the population of neural cells into the patient.

32. The method of claim 30 or 31, wherein the grafting comprises disrupting the patient’s blood-brain barrier.

33. The method of any one of claims 27-32, wherein the population of neural cells exhibits long-term survival after the disruption of the patient’s blood-brain barrier.

34. The method of any one of claims 27-33 wherein the population of neural cells exhibits long-term function after the disruption of the patient’s blood-brain barrier.

35. The method of any one of claims 27-34, wherein the population of neural cells maintains long-term survival in the patient after the patient experiences a subsequent disruption of the patient’s blood-brain barrier that is secondary to a neurological disorder or condition.

36. The method of any one of claims 27-35, wherein the population of neural cells maintains long-term function in the patient after the patient experiences a subsequent disruption of the patient’s blood-brain barrier that is secondary to a neurological disorder or condition.

37. The method of any one of claims 32-36, wherein the subsequent disruption of the patient’s blood-brain barrier is due to an infection or a stroke.

38. The method of any one of claims 27-37, wherein the population of neural cells survives and/or functions in the patient for at least one month, two months, three months, four months or more after administration.

39. The method of any one of claims 27-38, wherein the patient is not administered an immunosuppressive agent before administration of the population of neural cells.

40. The method of any one of claims 27-39, wherein the patient is not administered an immunosuppressive agent after administration of the population of neural cells.

41. The method of any one of claims 27-40, wherein the patient requires a reduced level of immunosuppression or is substantially free of immunosuppression.

42. The method of any one of claims 27-41, wherein the neural cell is selected from the group consisting of a cerebral endothelial cell, a neuron, an ependymal cell, an astrocyte, a microglial cell, an oligodendrocyte, a Schwann cell, a progenitor thereof, and a precursor thereof.

43. The neural cell of any one of claims 27-42, wherein the neural cell is a neural progenitor cell.

44. The neural cell of any one of claims 27-42, wherein the neural cell is a glial progenitor cell.

45. The neural cell of any one of claims 27-42, wherein the neural cell is a neuronal progenitor cell.

46. The method of any one of claims 27-45, wherein the microglial phagocytosis is associated with a neurological disorder or condition.

47. The method of any one of claims 35-46, wherein the neurological disorder or condition is selected from the group consisting of stroke, amyotrophic lateral sclerosis (ALS), cerebral hemorrhage, Parkinson’s disease, epilepsy, spinal cord injury, childhood hereditary leukodystrophies, congenital dysmyelination, Pelizaeus-Merzbacher disease, metabolic leukodystrophies, vanishing white matter disease, adrenoleukodystrophy, Canavan’s Disease, lysosomal storage diseases, Tay-Sachs disease, Sandhoff’s disease, Krabbe’s disease, Batten’s disease, metachromatic leukodystrophy, cerebral palsy, periventricular leukomalacia, spastic diplegias of prematurity, age-related white matter loss, subcortical dementia, vascular Leukoencephalopathies, subcortical stroke, diabetic leukoencephalopathy, hypertensive leukoencephalopathy, spinal cord injury, autoimmune demyelination, progressive multiple sclerosis, transverse myelitis, inflammatory demyelination, radiation toxicity, neurodegenerative diseases, Huntington’s Disease, frontotemporal dementia, and cerebrovascular disorders.

48. The method of any one of claims 35-46, wherein the neurological disorder or condition is Pelizaeus-Merzbacher disease.

49. The method of any one of claims 35-46, wherein the neurological disorder or condition is progressive multiple sclerosis.

50. The method of any one of claims 35-46, wherein the neurological disorder or condition is Huntington’s Disease.

51. The method of any one of claims 27-50, wherein the population of neural cells express CD47 at a higher level than in a parental pluripotent cell or in a unmodified neural cell.

52. The method of any one of claims 27-51, wherein the population of neural cells express a suicide gene that is activated by a trigger that causes the neural cell to die.

53. The method of any one of claims 27-52, wherein the population of neural cells are glial progenitor cells.

54. An in-vitro method for producing a therapeutically effective amount of a population of human neural cells from a population of human pluripotent stem cells comprising the steps of a) genetically modifying human pluripotent stem cells to i) reduce expression of MHC class I human leukocyte antigens and/or MHC class II human leukocyte antigens in the human pluripotent stem cells and ii) overexpress an exogenous CD47 polypeptide in the human pluripotent stem cells, b) differentiating the human pluripotent stem cells into neural cells; and c) assaying the neural cells for a hypoimmunogenicity phenotype and/or one or more neural cell-specific markers, gene expression, or gene expression profile.

55. The method of claim 54, wherein step a) further comprises genetically modifying human pluripotent stem cells to reduce expression of MHC class I and MHC class II human leukocyte antigens.

56. The method of claim 54, wherein step a) further comprises genetically modifying human pluripotent stem cells to reduce expression of MHC class I and MHC class II human leukocyte antigens.

57. The method of any one of claims 54-56, wherein the human pluripotent stem cells of step a)ii) express CD47 at a level higher than in the population of human pluripotent stem cells before step a).

58. The method of any one of claims 54-57, wherein the human neural cells of step b) or c) express CD47 at a level higher than in an unmodified neural cell or a neuronal cell not genetically modified by step a).

59. The method of any one of claims 54-56, wherein the human neural cells of step b) or c) have reduced expression of MHC class I human leukocyte antigens and/or MHC class II human leukocyte antigens compared to an unmodified human neural cell or a neuronal cell not genetically modified by step a).

60. The method of any one of claims 54-59, wherein step a) further comprises iii) express a suicide gene in the human pluripotent stem cells.

61. The method of any one of claims 54-60, wherein the assaying of the human neural cells in step c) comprises assaying for the hypoimmunogenicity phenotype by Elispot, ELISA, FACS, PCR, or mass cytometry (CYTOF).

62. An isolated neural cell comprising an exogenous CD47 polypeptide and reduced expression of MHC class I and/or class II human leukocyte antigens, wherein the cell evades immune recognition when administered to a patient.

63. The isolated neural cell of claim 62, wherein the isolated neuronal cell further comprises reduced expression of MHC class I and class II human leukocyte antigens.

64. The isolated neural cell of claim 62 or 63, wherein the isolated neural cell is selected from the group consisting of a cerebral endothelial cell, a neuron, an ependymal cell, an astrocyte, a microglial cell, an oligodendrocyte, a Schwann cell, a progenitor thereof, and a precursor thereof.

65. The isolated neural cell of any one of claims 62-64, wherein the isolated neural cell is a neural progenitor cell.

66. The isolated neural cell of any one of claims 62-64, wherein the isolated neural cell is a glial progenitor cell.

67. The isolated neural cell of any one of claims 62-64, wherein the isolated neural cell is a neuronal progenitor cell.

68. The isolated neural cell of any one of claims 62-64, wherein the isolated neural cell is a cerebral endothelial cell.

69. The isolated neural cell of any one of claims 62-64, wherein the isolated neural cell is a dopamine neuron.

70. The isolated neural cell of any one of claims 62-69, wherein the isolated neural cell evades immune recognition in vitro.

71. The isolated neural cell of any one of claims 62-70, wherein the isolated neural cell evades immune recognition when grafted into a patient’s central or peripheral nervous system.

72. The isolated neural cell of any one of claims 62-71, wherein the isolated neural cell undergoes, exhibits, or stimulates reduced microglial phagocytosis in vitro.

73. The isolated neural cell of any one of claims 62-72, wherein the isolated neural cell undergoes, exhibits, or stimulates reduced microglial phagocytosis when grafted into a patient’s central nervous system.

74. The isolated neural cell of any one of claims 62-73, wherein the isolated neural cell has reduced expression of B2M and/or CIITA.

75. The isolated neural cell of any one of claims 62-74, wherein the isolated neural cell comprises one or more CD47 transgenes.

76. The isolated neural cell of claim 75, wherein expression of the one or more CD47 transgene is controlled by constitutive promoters.

77. The isolated neural cell of claim 75, wherein expression of the one or more CD47 transgene is controlled by neuronal specific promoters.

78. A composition comprising a population of the isolated neural cells of any one of claims 62-77! Reference source not found.Error! Reference source not found.

79. The composition of claim 78, further comprising a pharmaceutically acceptable carrier.

80. A method for treating a neurological disorder or condition in a patient comprising administering to the patient a therapeutically effective amount of a population of neural cells comprising an exogenous CD47 polypeptide and reduced expression of MHC class I human leukocyte antigens and/or MHC class I human leukocyte antigens, wherein population of the neural cells undergoes, exhibits, or stimulates reduced microglial phagocytosis upon administration.

81. The method of claim 80, wherein the administering comprises grafting the population of neural cells into the patient’s central or peripheral nervous system.

82. The method of claim 81, wherein the grafting comprises injecting the population of neural cells into the patient.

83. The method of claim 81 or 82, wherein the grafting comprises disrupting the patient’s blood-brain barrier.

84. The method of any one of claims 80-83, wherein the population of neural cells exhibits long-term survival after the disruption of the patient’s blood-brain barrier.

85. The method of any one of claims 80-84, wherein the population of neural cells exhibits long-term function after the disruption of the patient’s blood-brain barrier.

86. The method of any one of claims 80-85, wherein the population of neural cells maintains long-term survival in the patient after the patient experiences a subsequent disruption of the patient’s blood-brain barrier that is secondary to the neurological disorder or condition.

87. The method of any one of claims 80-86, wherein the population of neural cells maintains long-term function in the patient after the patient experiences a subsequent disruption of the patient’s blood-brain barrier that is secondary to the neurological disorder or condition.

88. The method of any one of claims 80-87, wherein the subsequent disruption of the patient’s blood-brain barrier is due to an infection or a stroke.

89. The method of any one of claims 80-88, wherein the population of neural cells survives and/or functions in the patient for at least one month, two months, three months, four months or more after administration.

90. The method of any one of claims 80-89, wherein the patient is not administered an immunosuppressive agent before, during, and/or after administration of the population of neural cells.

91. The method of any one of claims 80-90, wherein the patient requires a reduced level of immunosuppression or is substantially free of immunosuppression.

92. The method of any one of claims 80-91, wherein the neural cell is selected from the group consisting of a cerebral endothelial cell, a neuron, an ependymal cell, an astrocyte, a microglial cell, an oligodendrocyte, a Schwann cell, a progenitor thereof, and a precursor thereof.

93. The method of any one of claims 80-92, wherein the neural cell is a neural progenitor cell.

94. The method of any one of claims 80-92, wherein the neural cell is a glial progenitor cell.

95. The method of any one of claims 80-92, wherein the neural cell is a neuronal progenitor cell.

96. The method of any one of claims 80-95, wherein the neurological disorder or condition is selected from the group consisting of stroke, amyotrophic lateral sclerosis (ALS), cerebral hemorrhage, Parkinson’s disease, epilepsy, spinal cord injury, childhood hereditary leukodystrophies, congenital dysmyelination, Pelizaeus-Merzbacher disease, metabolic leukodystrophies, vanishing white matter disease, adrenoleukodystrophy, Canavan’s Disease, lysosomal storage diseases, Tay-Sachs disease, Sandhoff’s disease, Krabbe’s disease, Batten’s disease, metachromatic leukodystrophy, cerebral palsy, periventricular leukomalacia, spastic diplegias of prematurity, age-related white matter loss, subcortical dementia, vascular Leukoencephalopathies, subcortical stroke, diabetic leukoencephalopathy, hypertensive leukoencephalopathy, spinal cord injury, autoimmune demyelination, progressive multiple sclerosis, transverse myelitis, inflammatory demyelination, radiation toxicity, neurodegenerative diseases, Huntington’s Disease, frontotemporal dementia, and cerebrovascular disorders.

97. The method of any one of claims 80-95, wherein the neurological disorder or condition is Pelizaeus-Merzbacher disease.

98. The method of any one of claims 80-95, wherein the neurological disorder or condition is progressive multiple sclerosis.

99. The method of any one of claims 80-95, wherein the neurological disorder or condition is Huntington’s Disease.

100. A neural cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the neural cell undergoes, exhibits, or stimulates reduced microglial phagocytosis.

101. The neural cell of claim 100, wherein the neural cell is selected from the group consisting of a cerebral endothelial cell, a neuron, an ependymal cell, an astrocyte, a microglial cell, an oligodendrocyte, a Schwann cell, a progenitor thereof, and a precursor thereof.

102. The neural cell of any one of claims 100-101, wherein the neural cell is a neural progenitor cell.

103. The neural cell of any one of claims 100-101, wherein the neural cell is a glial progenitor cell.

104. The neural cell of any one of claims 100-101, wherein the neural cell is a neuronal progenitor cell.

105. A cerebral endothelial cell in vitro differentiated from a stem cell expressing an exogenous CD47 polypeptide and expressing i) reduced expression levels of MHC class I and/or II human leukocyte antigens and/or ii) reduced expression levels of B2M and/or CIITA, wherein the cerebral endothelial cell undergoes, exhibits, or stimulates reduced microglial phagocytosis.

106. The cerebral endothelial cell of claim 105, wherein the cell forms vasculature when administered to a patient’s brain.

107. A microglial cell in vitro differentiated from a stem cell expressing an exogenous CD47 polypeptide and expressing: i)reduced expression levels of MHC class I and/or II human leukocyte antigens and/or ii) reduced expression levels of B2M and/or CIITA, wherein the microglial cell undergoes, exhibits, or stimulates reduced microglial phagocytosis.

108. An oligodendrocyte in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the oligodendrocyte undergoes, exhibits, or stimulates reduced microglial phagocytosis.

109. A Schwann cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the Schwann cell undergoes, exhibits, or stimulates reduced microglial phagocytosis.

110. An astrocyte in vitro differentiated from a stem cell expressing an exogenous CD47 polypeptide and expressing: i)reduced expression levels of MHC class I and/or II human leukocyte antigens and/or ii) reduced expression levels of B2M and/or CIITA, wherein the astrocyte undergoes, exhibits, or stimulates reduced microglial phagocytosis.

111. An ependymal cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the ependymal cell undergoes, exhibits, or stimulates reduced microglial phagocytosis.

112. A neuron in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

113. The neuron of claim 112, wherein the neuron is a dopamine neuron.

114. A dopamine neuron in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

115. An isolated neural cell comprising an exogenous CD24 polypeptide and reduced expression of MHC class I human leukocyte antigens and/or MHC class II human leukocyte antigens, wherein the isolated neural cell evades immune recognition when administered to a patient.

116. The isolated neural cell of claim 115, wherein the isolated neural cell is selected from the group consisting of a cerebral endothelial cell, a neuron, an ependymal cell, an astrocyte, a microglial cell, an oligodendrocyte, a Schwann cell, a progenitor thereof, and a precursor thereof.

117. The isolated neural cell of claim 115 or 116, wherein the isolated neural cell is a neural progenitor cell.

118. The isolated neural cell of claim 115 or 116, wherein the isolated neural cell is a glial progenitor cell.

119. The isolated neural cell of claim 115 or 116, wherein the isolated neural cell is a neuronal progenitor cell.

120. The isolated neural cell of claim 115 or 116, wherein the neural cell is a cerebral endothelial cell.

121. The isolated neural cell of any one of claims 115-120, wherein the neural cell evades immune recognition in vitro.

122. The isolated neural cell of any one of claims 115-121, wherein the neural cell evades immune recognition when grafted into a patient’s central or peripheral nervous system.

123. The isolated neural cell of any one of claims 115-122, wherein the neural cell undergoes, exhibits, or stimulates reduced microglial phagocytosis in vitro.

124. The isolated neural cell of any one of claims 115-123, wherein the neural cell undergoes, exhibits, or stimulates reduced microglial phagocytosis when grafted into a patient’s central nervous system.

125. The isolated neural cell of any one of claims 115-124, wherein the neural cell has reduced expression of B2M and/or CIITA.

126. The isolated neural cell of any one of claims 115-125, wherein the neural cell comprises one or more CD24 transgenes.

127. The isolated neural cell of claim 126, wherein expression of the one or more CD47 transgene is controlled by constitutive promoters.

128. The isolated neural cell of claim 126, wherein expression of the one or more CD47 transgene is controlled by neuronal specific promoters.

129. A composition comprising a population of the isolated neural cells of any one of claims 115-128.

130. The composition of claim 129, further comprising a pharmaceutically acceptable carrier.

131. A method for treating a neurological disorder or condition in a patient comprising administering to the patient a therapeutically effective amount of a population of neural cells comprising an exogenous CD24 polypeptide and reduced expression of MHC class I and/or MHC class II human leukocyte antigens.

132. The method of claim 131, further comprises reduced expression of MHC class I and MHC class II human leukocyte antigens.

133. The method of claim 131 or 132, wherein the administering comprises grafting the population of neural cells into the patient’s central or peripheral nervous system.

134. The method of claim 133, wherein the grafting comprises injecting the population of neural cells into the patient.

135. The method of claim 133 or 134, wherein the grafting comprises disrupting the patient’s blood-brain barrier.

136. The method of any one of claims 131-135, wherein the population of neural cells maintains long-term survival after the disruption of the patient’s blood-brain barrier.

137. The method of any one of claims 131-136, wherein the population of neural cells maintains long-term function after the disruption of the patient’s blood-brain barrier.

138. The method of any one of claims 131-137, wherein the population of neural cells maintains long-term survival in the patient after the patient experiences a subsequent disruption of the patient’s blood-brain barrier that is secondary to the neurological disorder or condition.

139. The method of any one of claims 131-138, wherein the population of neural cells maintains long-term function in the patient after the patient experiences a subsequent disruption of the patient’s blood-brain barrier that is secondary to the neurological disorder or condition.

140. The method of any one of claims 131-139, wherein the subsequent disruption of the patient’s blood-brain barrier is due to an infection or a stroke.

141. The method of any one of claims 131-140, wherein the population of neural cells survives and/or functions in the patient for at least one month, two months, three months, four months or more after administration.

142. The method of any one of claims 131-141, wherein the patient is not administered an immunosuppressive agent before administration of the population of neural cells.

143. The method of any one of claims 131-142, wherein the patient is not administered an immunosuppressive agent after administration of the population of neural cells.

144. The method of any one of claims 131-143, wherein the patient requires a reduced level of immunosuppression or is substantially free of immunosuppression.

145. The method of any one of claims 131-144, wherein the neural cell is selected from the group consisting of a cerebral endothelial cell, a neuron, an ependymal cell, an astrocyte, a microglial cell, an oligodendrocyte, a Schwann cell, a progenitor thereof, and a precursor thereof.

146. The method of any one of claims 131-144, wherein the neural cell is a neural progenitor cell.

147. The method of any one of claims 131-144, wherein the neural cell is a glial progenitor cell.

148. The method of any one of claims 131-144, wherein the neural cell is a neuronal progenitor cell.

149. The method of any one of claims 131-148, wherein the neurological disorder or condition is selected from the group consisting of stroke, amyotrophic lateral sclerosis (ALS), cerebral hemorrhage, Parkinson’s disease, epilepsy, spinal cord injury, childhood hereditary leukodystrophies, congenital dysmyelination, Pelizaeus-Merzbacher disease, metabolic leukodystrophies, vanishing white matter disease, adrenoleukodystrophy, Canavan’s Disease, lysosomal storage diseases, Tay-Sachs disease, Sandhoff’s disease, Krabbe’s disease, Batten’s disease, metachromatic leukodystrophy, cerebral palsy, periventricular leukomalacia, spastic diplegias of prematurity, age-related white matter loss, subcortical dementia, vascular Leukoencephalopathies, subcortical stroke, diabetic leukoencephalopathy, hypertensive leukoencephalopathy, spinal cord injury, autoimmune demyelination, progressive multiple sclerosis, transverse myelitis, inflammatory demyelination, radiation toxicity, neurodegenerative diseases, Huntington’s Disease, frontotemporal dementia, and cerebrovascular disorders.

150. The method of any one of claims 131-148, wherein the neurological disorder or condition is Pelizaeus-Merzbacher disease.

151. The method of any one of claims 131-148, wherein the neurological disorder or condition is progressive multiple sclerosis.

152. The method of any one of claims 131-148, wherein the neurological disorder or condition is Huntington’s Disease.

153. A neural cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neural cell undergoes, exhibits, or stimulates reduced microglial phagocytosis.

154. The neural cell of claim 153, wherein the neural cell is selected from the group consisting of a cerebral endothelial cell, a neuron, an ependymal cell, an astrocyte, a microglial cell, an oligodendrocyte, a Schwann cell, a progenitor thereof, and a precursor thereof.

155. The neural cell of claim 153 or 154, wherein the neural cell is a neural progenitor cell.

156. The neural cell of claim 153 or 154, wherein the neural cell is a glial progenitor cell.

157. The neural cell of claim 153 or 154, wherein the neural cell is a neuronal progenitor cell.

158. A cerebral endothelial cell in vitro differentiated from a stem cell expressing an exogenous CD24 polypeptide and expressing: i) reduced expression levels of MHC class I and II human leukocyte antigens and/or ii) reduced expression levels of B2M and/or CIITA, wherein the cerebral endothelial cell undergoes, exhibits, or stimulates reduced microglial phagocytosis.

159. The cerebral endothelial cell of claim 158, wherein the cell forms vasculature when administered to a patient’s brain.

160. A microglial cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the microglial cell undergoes, exhibits, or stimulates reduced microglial phagocytosis.

161. An oligodendrocyte in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the oligodendrocyte undergoes, exhibits, or stimulates reduced microglial phagocytosis.

162. A Schwann cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the Schwann cell undergoes, exhibits, or stimulates reduced microglial phagocytosis.

163. An astrocyte in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the astrocyte undergoes, exhibits, or stimulates reduced microglial phagocytosis.

164. An ependymal cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the ependymal cell undergoes, exhibits, or stimulates reduced microglial phagocytosis.

165. A neuron in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

166. The neuron of claim 165, wherein the neuron is a dopamine neuron.

167. A dopamine neuron in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

168. Use of a neural cell to treat a neurological disease, comprising an isolated neural cell comprising an exogenous CD47 polypeptide and reduced expression of MHC class I and/or class II human leukocyte antigens, wherein the cell evades immune recognition when administered to a patient.

169. Use of a neural cell to treat a neurological disease, comprising a neural cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the neural cell undergoes, exhibits, or stimulates reduced microglial phagocytosis.

170. Use of a neural cell to treat a neurological disease, comprising a cerebral endothelial cell in vitro differentiated from a stem cell expressing an exogenous CD47 polypeptide and expressing i) reduced expression levels of MHC class I and/or II human leukocyte antigens and/or ii) reduced expression levels of B2M and/or CIITA, wherein the cerebral endothelial cell undergoes, exhibits, or stimulates reduced microglial phagocytosis.

171. Use of a neural cell to treat a neurological disease, comprising a microglial cell in vitro differentiated from a stem cell expressing an exogenous CD47 polypeptide and expressing: i)reduced expression levels of MHC class I and/or II human leukocyte antigens and/or ii) reduced expression levels of B2M and/or CIITA, wherein the microglial cell undergoes, exhibits, or stimulates reduced microglial phagocytosis.

172. Use of a neural cell to treat a neurological disease, comprising an oligodendrocyte in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the oligodendrocyte undergoes, exhibits, or stimulates reduced microglial phagocytosis.

173. Use of a neural cell to treat a neurological disease, comprising a Schwann cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the Schwann cell undergoes, exhibits, or stimulates reduced microglial phagocytosis.

174. Use of a neural cell to treat a neurological disease, comprising an astrocyte in vitro differentiated from a stem cell expressing an exogenous CD47 polypeptide and expressing: i)reduced expression levels of MHC class I and/or II human leukocyte antigens and/or ii) reduced expression levels of B2M and/or CIITA, wherein the astrocyte undergoes, exhibits, or stimulates reduced microglial phagocytosis.

175. Use of a neural cell to treat a neurological disease, comprising an ependymal cell in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the ependymal cell undergoes, exhibits, or stimulates reduced microglial phagocytosis.

176. Use of a neural cell to treat a neurological disease, comprising a neuron in vitro differentiated from a stem cell expressing an exogenous CD47 polypeptide and expressing: i)reduced expression levels of MHC class I and/or II human leukocyte antigens and/or ii) reduced expression levels of B2M and/or CIITA, wherein the neuron undergoes, exhibits, or stimulates reduced microglial phagocytosis.

177. Use of a neural cell to treat a neurological disease, comprising a dopamine neuron in vitro differentiated from a stem cell expressing an exogenous CD47 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

178. Use of a neural cell to treat a neurological disease, comprising an isolated neural cell comprising an exogenous CD24 polypeptide and reduced expression of MHC class I human leukocyte antigens and/or MHC class II human leukocyte antigens, wherein the isolated neural cell evades immune recognition when administered to a patient.

179. Use of a neural cell to treat a neurological disease, comprising a neural cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neural cell undergoes, exhibits, or stimulates reduced microglial phagocytosis.

180. Use of a neural cell to treat a neurological disease, comprising a cerebral endothelial cell in vitro differentiated from a stem cell expressing an exogenous CD24 polypeptide and expressing: i) reduced expression levels of MHC class I and II human leukocyte antigens and/or ii) reduced expression levels of B2M and/or CIITA, wherein the cerebral endothelial cell undergoes, exhibits, or stimulates reduced microglial phagocytosis.

181. Use of a neural cell to treat a neurological disease, comprising a microglial cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the microglial cell undergoes, exhibits, or stimulates reduced microglial phagocytosis.

182. Use of a neural cell to treat a neurological disease, comprising an oligodendrocyte in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the oligodendrocyte undergoes, exhibits, or stimulates reduced microglial phagocytosis.

183. Use of a neural cell to treat a neurological disease, comprising a Schwann cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA,, wherein the Schwann cell undergoes, exhibits, or stimulates reduced microglial phagocytosis.

184. Use of a neural cell to treat a neurological disease, comprising an astrocyte in vitro differentiated from a stem cell expressing an exogenous CD24 polypeptide and expressing: i) reduced expression levels of MHC class I and/or II human leukocyte antigens and/or ii) reduced expression levels of B2M and/or CIITA,, wherein the astrocyte exhibits undergoes, exhibits, or stimulates reduced microglial phagocytosis.

185. Use of a neural cell to treat a neurological disease, comprising an ependymal cell in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the ependymal cell undergoes, exhibits, or stimulates reduced microglial phagocytosis.

186. Use of a neural cell to treat a neurological disease, comprising a neuron in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

187. Use of a neural cell to treat a neurological disease, comprising a dopamine neuron in vitro differentiated from a stem cell expressing an exogenous CD24 polypeptide and expressing: i) reduced expression levels of MHC class I and/or II human leukocyte antigens and/or ii) reduced expression levels of B2M and/or CIITA, wherein the neuron undergoes, exhibits, or stimulates reduced microglial phagocytosis.

188. The use of any one of claims 168-187, wherein the neurological disorder or condition is selected from the group consisting of stroke, amyotrophic lateral sclerosis (ALS), cerebral hemorrhage, Parkinson’s disease, epilepsy, spinal cord injury, childhood hereditary leukodystrophies, congenital dysmyelination, Pelizaeus-Merzbacher disease, metabolic leukodystrophies, vanishing white matter disease, adrenoleukodystrophy, Canavan’s Disease, lysosomal storage diseases, Tay-Sachs disease, Sandhoff’s disease, Krabbe’s disease, Batten’s disease, metachromatic leukodystrophy, cerebral palsy, periventricular leukomalacia, spastic diplegias of prematurity, age-related white matter loss, subcortical dementia, vascular Leukoencephalopathies, subcortical stroke, diabetic leukoencephalopathy, hypertensive leukoencephalopathy, spinal cord injury, autoimmune demyelination, progressive multiple sclerosis, transverse myelitis, inflammatory demyelination, radiation toxicity, neurodegenerative diseases, Huntington’s Disease, frontotemporal dementia, and cerebrovascular disorders.

189. The use of any one of claims 165-187, wherein the neurological disorder or condition is Pelizaeus-Merzbacher disease.

190. The use of any one of claims 165-187, wherein the neurological disorder or condition is progressive multiple sclerosis.

191. The use of any one of claims 165-187, wherein the neurological disorder or condition is Huntington’s Disease.

192. A dopamine neuron in vitro differentiated from a stem cell, wherein the dopamine neuron expresses an exogenous CD47 polypeptide, wherein the dopamine neuron has reduced expression levels of B2M and CIITA, and wherein the dopamine neuron undergoes, exhibits, or stimulates reduced microglial phagocytosis.

193. A dopamine neuron in vitro differentiated from a stem cell, wherein the dopamine neuron expresses an exogenous CD24 polypeptide, wherein the dopamine neuron has reduced expression levels of B2M and CIITA, and wherein the dopamine neuron undergoes, exhibits, or stimulates reduced microglial phagocytosis.

194. A glial progenitor cell in vitro differentiated from a stem cell, wherein the glial progenitor cell expresses an exogenous CD47 polypeptide, wherein the glial progenitor cell has reduced expression levels of B2M and CIITA, and wherein the glial progenitor cell undergoes, exhibits, or stimulates reduced microglial phagocytosis or evades microglial phagocytosis.

195. A glial progenitor cell in vitro differentiated from a stem cell, wherein the glial progenitor cell expresses an exogenous CD24 polypeptide, wherein the glial progenitor cell has reduced expression levels of B2M and CIITA, and wherein the glial progenitor cell undergoes, exhibits, or stimulates reduced microglial phagocytosis or evades microglial phagocytosis.
